(19) United States
(12) Patent Application Publication
(10) Pub. No.: US 2010/0280059 A1
(43) Pub. Date: Nov. 4, 2010

(54) COMPOSITIONS COMPRISING AN ANTIHISTAMINE, ANTITUSSIVE AND DECONGESTANT IN EXTENDED RELEASE FORMULATIONS

(75) Inventors: James Joseph McDermott, Ashland, VA (US); R. Gary Hollenbeck, Ellicott City, MD (US); Craig Linwood Attkisson, Ashland, VA (US)

Correspondence Address: FOLEY AND LARDNER LLP;SUITE 500, 3000 K STREET NW, WASHINGTON, DC, 20007, US

(73) Assignee: Atley Pharmaceuticals, Inc.

(21) Appl. No.: 12/770,484

(22) Filed: Apr. 29, 2010

Related U.S. Application Data
(60) Provisional application No. 61/174,912, filed on May 1, 2009.

(51) Int. Cl. A61K 31/485; A61P 11/00; A61P 11/02; A61P 11/14

(52) U.S. Cl. 514/282

(57) ABSTRACT

The invention provides oral formulations for the treatment of cold and allergy symptoms. Each formulation combines an antihistamine, an antitussive, and/or a decongestant into one extended release composition. The invention further provides for methods of making and using such formulations, as well as for methods for preventing abuse or extraction of a single drug present in an oral extended release composition comprising two or more of an antihistamine, antitussive, and/or decongestant.

### CROSS-REFERENCE TO RELATED APPLICATION

This application claims priority from U.S. Provisional Patent Application No. 61/174,912, filed May 1, 2009. The contents of that application are incorporated herein by reference in its entirety.

Americans suffer an estimated one billion colds a year, 2 to 4 colds per year for adults and 6 to 10 colds a year for children. National Institute of Allergy and Infectious Diseases (NIAID) Facts Sheets. “The Common Cold,” available at www.niaid.nih.gov/factsheets/cold.htm December 2004. Approximately nine out of ten Americans will have at least one cold or similar form of upper respiratory infection annually. Colds are the most prevalent illness in children, occurring more frequently then all other diseases combined and accounting for as much as 50% of all school absenteeism. Micromedex Healthcare Series, “The Common Cold Etiology and Treatment,” available at www.thomsonhc.com/hcs/librarian/ND, accessed Jun. 18, 2008. Colds occur most frequently during the colder months spanning from August or early September through March or April. The lower humidity that tends to accompany decreased ambient temperatures may combine with the increased indoor and close quarter person to person interaction to enhance the proliferation of viruses that cause colds. National Institute of Allergy and Infectious Diseases (NIAID) Facts Sheets “The Common Cold” available at www.niaid.nih.gov/factsheets/cold.htm, accessed December 2004.

### BACKGROUND OF THE INVENTION

Americans suffer an estimated one billion colds a year, 2 to 4 colds per year for adults and 6 to 10 colds a year for children. National Institute of Allergy and Infectious Diseases (NIAID) Facts Sheets. “The Common Cold,” available at www.niaid.nih.gov/factsheets/cold.htm December 2004. Approximately nine out of ten Americans will have at least one cold or similar form of upper respiratory infection annually. Colds are the most prevalent illness in children, occurring more frequently then all other diseases combined and accounting for as much as 50% of all school absenteeism. Micromedex Healthcare Series, “The Common Cold Etiology and Treatment,” available at www.thomsonhc.com/hcs/librarian/ND, accessed Jun. 18, 2008. Colds occur most frequently during the colder months spanning from August or early September through March or April. The lower humidity that tends to accompany decreased ambient temperatures may combine with the increased indoor and close quarter person to person interaction to enhance the proliferation of viruses that cause colds. National Institute of Allergy and Infectious Diseases (NIAID) Facts Sheets “The Common Cold” available at www.niaid.nih.gov/factsheets/cold.htm, accessed December 2004.

Patients with the common cold typically present with signs and symptoms of nasal discharge, obstruction of nasal breathing, swelling of the sinus membranes, sneezing, sore throat, cough and headache. Micromedex Healthcare Series, “The Common Cold Etiology and Treatment,” ibid. Seventy to 90% of patients suffer from rhinorrhea or sneezing, 65% of patients have nasal obstruction or congestion or and 25% of patients have cough or hoarseness. Id. Most colds last between 7-14 days. Patients experiencing symptoms longer than 2 weeks may also be affected by allergies. Id.

According to the American Lung Association, colds account for more visits to the doctor than any other condition. American Lung Association, “Cold and Flu Guidelines: The Common Cold,” available at www.lungusa.org, accessed Jun. 16, 2008. In fact, a study on the impact of the common cold showed that in 2003 alone there were more than 100 million physician visits related to colds, at a cost of $7.7 billion. The study also estimated that children missed 189 million days of school annually and parents missed 126 million days of work to care for a child with a cold. WebMD. “Cost of the Common Cold: $40 Billion” available at www.medmutual.com, accessed Feb. 24, 2003; Fendrick, et al., “The Economic Burden of Non-Influenza-Related Viral Respiratory Tract Infection in the United States.” Arch Intern Med. 163(4): 487-494 (2003). When added to the 150 million workdays missed by employees suffering from a cold, the total economic impact of cold-related work loss exceeds $20 billion. Fendrick, ibid.; Garibaldi R A, “Epidemiology of community-acquired respiratory tract infections in adults. Incidence, etiology, and impact” Am. J. Med. 78 (6B): 32-37 (1985); Common Cold. National Institute of Allergy and Infectious Diseases www3.niaid.nih.gov/healthscience/healthtopics/colds/ Retrieved on Jun. 11, 2008; US Census Bureau. www.Quickfacts.census.gov/qfd/states/00000.html 2004 Estimates.

American spending on over-the-counter (OTC) and prescription drugs for cough and cold relief is in excess of $3 billion annually. WebMD, ibid; Fendrick, ibid. In the USA alone, the common cold leads to 75 to 100 million physician visits annually at a conservative cost estimate of $7.7 billion per year. Americans spend $2.9 billion on over-the-counter drugs and another $400 million on prescription medicines for symptomatic relief. Fendrick., ibid; Garibaldi, ibid. More than one-third of patients who saw a doctor received an antibiotic prescription, which not only contributes to unnecessary costs ($1.1 billion annually on an estimated 41 million antibiotic prescriptions in the United States), but also has implications for antibiotic resistance from overuse of such drugs. Fendrick, ibid. According to IMS Health (August 2007), annual prescriptions for cough, cold and flu medicines were approximately 42,858,000. Because no single active pharmaceutical ingredient (API) treats all cold symptoms, combination products often provide a convenient and sometimes less expensive means of providing relief than the use of multiple single-ingredient products.

The present invention provides unique benefits as compared to immediate release and/or combination cold and allergy products that are current available. For example, in one embodiment, the present invention integrates the benefits of three APIs, i.e., an antihistamine (e.g., chlorpheniramine), an antitussive (e.g., hydrocodone), and a decongestant (e.g., pseudoephedrine), into one extended release (ER) (e.g., 12 hour) composition. Previously, when used together, these APIs needed to be dosed 4-6 times daily in their immediate release (IR) forms because they are not currently available in a single extended release triple-acting combination product.

Extended release products do already exist on the market that contain one or two of the APIs, as well as some IR products that contain all three drugs. For example, marketed OTC or prescription drugs containing chlorpheniramine (CPM), hydrocodone (HC), and/or pseudoephedrine (PSE) in IR or ER forms include the following:

Products with Pseudoephedrine
Afrinol®
Cenafed®
D-Isoephedrine
D-Pseudoephedrine
Decofed®
Dimetapp® Decongestant
Dimetapp® Decongestant Pediatric Drops
Drixoral® Nasal Decongestant
Efidac 24® Pseudoephedrine HCl
Eltor 120®
Genaphed®
Isoephedrine
Maxenal®
Myfedrine®
Novafed®
Pedia Care®
Pseudo 60's®
Pseudo-12®
Pseudoephdrine HCl
Sudafed 12 Hour®
Sudafed 24 Hour®
Sudogest
   Products with Chlorpheniramine
Aller-Chlor®
Antagonate®
Chlo-Amine®
Chlor-Trimeton®
Chlor-Tripolon®
Dexchlorpheniramine Maleate
Efidac 24® Chlorpheniramine Maleate
Gen-Allerate®
Haynon®
Histadur®
Kloromin®
Mylaramine®
Novo-Pheniram®
Phenetron®
Piriton®
Polaramine®
Pyridamal 100®
Telachlor®
Teldrin®
   Products with Hydrocodone
Hycodan® (includes homatropine methylbromide)
Lortab® (includes acetaminophen)
Maxidone® (includes acetaminophen)
Norco® (includes acetaminophen)
Vicodin® (includes acetaminophen)
Zydone® (includes acetaminophen)
   Products with Pseudoephedrine+Chlorpheniramine:
Allerest®
Anamine®
Biohist-LA®
Brexin®
Chlordrine® SR
Chlor-Phed®
Chlor-Trimeton® (4 hour and 12 hour)
Deconamine®
De-congestine TR®
Dynahist ER®
Histalet® Syrup
Kronofed-A® Kronocaps
Kronofed-A-Jr.® Kronocaps
ND® Clear
Pseudoephed/Chlorphen 100®
Rescon®
Rescon® Jr.
Rescon® ED
Ryna®
Sudafed® Cold and Allergy
Tanafed®
   Products with Chlorpheniramine+Hydrocodone
Tussionex® Pennkinetic® Extended Release Suspension
TussiCaps® Extended Release Capsules
S-T Forte® 2
   Products with Pseudoephedrine+Hydrocodone
Detussin® Liquid
Histussin® D Liquid
Tyrodone® Liquid
   Products with Chlorpheniramine+Pseudoephedrine+Hydrocodone
A-G Tussin®
Atuss® HD
Cordron-HC®
Hexatussin®
Histinex® PV
Hydrocof-HC®
Hydron® PCS
Hydrotuss® HC
Hyphed®
KG-Tussin®
M-End®
Notuss®
P-V-Tussin® Syrup
Pediatex® HC
Q-V Tussin®
Tussin-V®

None of the above-mentioned triple-acting formulations (containing CPM, PSE, and HC in a single product) are extended release products for all three drugs. Likewise, no group has established that administration to a patient of a single dose of an oral composition comprising all three active ingredients provides serum levels of the three drugs over 12 hours that are bioequivalent to serum levels achieved upon administration of an appropriate number of doses over 12 hours of FDA-approved immediate release reference listed drug (RLD) compositions comprising the active ingredients.

Similarly, none of the above-mentioned formulations containing hydrocodone and pseudoephedrine (with or without any other active ingredient, such as CMP) are extended release products. No group has established that administration to a patient of a single dose of an oral composition comprising hydrocodone and pseudoephedrine provides serum levels of these two active ingredients over 12 hours that are bioequivalent to serum levels achieved upon administration of an appropriate number of doses over 12 hours of FDA-approved immediate release RLD compositions comprising hydrocodone and/or pseudoephedrine. In fact, no products containing both hydrocodone and pseudoephedrine—with or without another active ingredient, immediate or extended release—are currently FDA approved. It should be noted that the foregoing list of marketed drugs includes drugs that are not FDA-approved, but are nonetheless marketed.

As relevant to other embodiments of the present invention, diversion and abuse of drugs present in OTC and prescribed products has escalated in recent years. For example, many OTC cold and allergy tablets contain pseudoephedrine or ephedrine, which is used to clandestinely produce a drug of abuse known as methamphetamine. This drug, also known as “meth,” “speed,” “crank,” or “ice,” is a powerful, addictive stimulant that affects the central nervous system. Methamphetamine is sold illegally in the form of pills, capsules, or powder that can be smoked, snorted, injected, or swallowed.

Makeshift secret and illegal laboratories (“meth labs”) isolate pseudoephedrine or ephedrine from OTC cold and allergy tablets using a solution of water, alcohol, or other solvent for several hours until the pseudoephedrine or ephedrine separates from the tablet. The pseudoephedrine or ephedrine is then be converted into methamphetamine using readily available common household products and easily assessable equipment, such as alcohol, Coleman fuel, acetone, road flares, drain cleaners, iodine, muriatic acid, rock salt, starting fluid, coffee filters and matches. Methods for making the drug are available from “recipes” and exchange of information readily available via the Internet.

Methamphetamine trafficking and abuse are on the rise in the United States, and measures to prevent theft and diversion of pseudoephedrine are cumbersome and costly.

Many other OTC and prescription products, containing drugs such as opioids, are also ripe for diversion for illegal drug abuse. For example, hydrocodone is legally used as an antitussive (cough suppressant) in cold medicines, and as analgesic agent for the treatment of moderate to moderately severe pain in prescription medicines. Hydrocodone is the most frequently prescribed opiate in the U.S. with over 110 million prescriptions for hydrocodone-containing products dispensed in 2003. Although it is generally not clandestinely produced, hydrocodone is currently illegally diverted and directly abused for its euphoria and pain-relieving effects. Widespread diversion occurs via bogus call-in prescriptions, altered prescriptions, theft and illicit purchases from Internet sources.

Thus, a need exists for preparing and selling OTC and prescription products that avoid the potential for abuse and diversion of drugs for illegal use. For example, a need exists for cold/cough and allergy formulations where drugs such as pseudoephedrine, ephedrine and/or hydrocodone cannot easily be extracted or separated out in a makeshift laboratory. Likewise, a need exists for cold/cough, pain relief and muscle relaxation products that cannot be easily diverted for illegal purposes, regardless of whether they are clandestinely produced in illegal laboratories.

### SUMMARY OF THE INVENTION

Embodiments of the present invention overcome problems and disadvantages previously associated with formulations of immediate release (IR) and/or combination products comprising one or more of the following active ingredients: (1) an antihistamine, (2) an antitussive, and (3) a decongestant. In contrast to the many products currently commercially available, the present invention provides a formulation that allows extended release (ER) (e.g., 12 hour) of all three active ingredients. As one example, the present invention provides an oral formulation that comprises a novel mixture of IR and ER forms of chlorpheniramine (an antihistamine), hydrocodone (a narcotic antitussive) and pseudoephedrine (a decongestant), in a single product. This formulation results in an ER combination product that can be dosed twice daily with the same effectiveness as previously available IR forms (which have been sold and administered either individually or via combination products). The formulation is also superior to existing single and combination ER formulations in that it (a) provides both IR and ER dosages of drugs for immediate and long term drug delivery; (b) provides bioequivalent dosages of three RLDs in a single dosage form and (c) resists abuse and diversion of the component drugs.

In one embodiment, the present invention provides that administration to a patient of a single dose of an oral drug composition comprising decongestant, antitussive and/or antihistamine active ingredients provides serum levels of those active ingredients over 12 hours that are bioequivalent to serum levels achieved upon administration of an appropriate number of doses over 12 hours of FDA-approved immediate release reference listed drug (IR RLD) compositions comprising the active ingredients. The appropriate number of doses of the IR RLDs corresponds to the number of doses recommended in one or more FDA-approved labels for the administration of the IR RLDs over 12 hours.

In another embodiment, administration to a patient of a sufficient number of doses of an extended release oral composition comprising decongestant, antitussive and/or antihistamine active ingredients to achieve steady-state serum levels of the active ingredients over a dosing period of greater than 24 hours yields serum levels of those active ingredients that are bioequivalent to serum levels achieved upon administration of an appropriate number of doses over the same time period of one or more FDA-approved IR drug compositions comprising the active ingredients. The appropriate number of doses of IR drugs corresponds to a number of doses recommended in one or more FDA-approved labels for the administration of the one or more FDA-approved IR drugs over the same time period. In another embodiment, the present invention provides oral formulations comprising hydrocodone and pseudoephedrine, with or without an antihistamine, that allow for extended release of those active ingredients in a human.

In one embodiment, the ER portion of the present invention formulation may exist in the form of coated beads, particulates or pellets within a liquid suspension, with an IR portion in the liquid suspension. Alternatively, the ER portion may comprise a solid dosage form such as capsule, tablet, or other oral solid, with an IR portion as a secondary layer or medium outside the ER portion. In one embodiment, the product is formulated to be dosed once every 12 hours. Other embodiments include those dosed every 8 hours, 16 hours, 24 hours, etc.

Likewise, in another embodiment, a single combination ER product exhibits a specific IR to ER ratio, where the ER component is in a particulate, pellet, or bead and the IR portion is outside the particulates, pellets, or beads (e.g., suspended in syrup; in powder in a capsule, tablet, etc.). The ratio achieves blood serum levels that are bioequivalent (BE) to reference listed drugs (RLDs) at both single-dose and steady state conditions. In other embodiments, one using the present formulations obtains certain specific blood serum ranges (as measured by AUC, Tₘₐₓ, T_{1/2}, etc.) in humans over time, where the levels are bioequivalent to RLDs at both single-dose and steady state conditions.

In one embodiment, the present invention relates to a method for making oral extended release drug compositions comprising a first portion comprising an antihistamine, an antitussive, and optionally a decongestant, as active ingredients in an immediate release form, and a second portion comprising particulates, pellets or beads that comprises the antihistamine, the antitussive and the decongestant as active ingredients in an extended release form. In another embodiment, a method of the present invention involves making a composition so that an IR portion initially comprises an antihistamine and an antitussive, but not a decongestant, and an ER portion comprises an antihistamine, an antitussive and pseudoephedrine. In a related embodiment, leaching of one or more of the ER components, such as pseudoephedrine, into the IR vehicle may be used to provide the IR form of one or more components.

In another embodiment, the present formulation provides particles that comprise all three drugs in a single bead. Such an embodiment offers advantages over previously available ER formulations that provide each drug in separate beads in terms of both dosage efficacy, safety, and resistance to diversion and abuse.

In other embodiments of the present invention, formulations comprise ER particulates, pellets or beads that comprise pseudoephedrine (or chemically related decongestant, such as ephedrine) and/or narcotic antitussives (such as hydrocodone) in a manner that prevents or makes difficult the misuse, abuse or illegal diversion of such drugs, as compared to other commercially available OTC or prescription products comprising one or more of these drugs. For example, if each individual ER particulate, pellet or bead in the present formulations comprises pseudoephedrine (or related compound) along with other compounds, including an antihistamine and antitussive, such as chlorpheniramine and hydrocodone, the formulations will prevent or make difficult the separation or extraction of PSE (or ephedrine) and/or a narcotic antitussive from the final formulation for the purpose of preparing meth or isolating a narcotic.

While it may be technically possible to extract PSE, ephedrine and/or a narcotic from formulations of the present invention (e.g. from the beads), extraction will require elaborate and expensive equipment and techniques. Most illegal producers of meth will not have access to such equipment and/or resources to make extraction worthwhile, especially as compared what can be easily done using other OTC products containing these drugs. Thus, unlike many commercially available OTC formulations containing PSE (or related compound) or a narcotic antitussive, retailers will be able to sell OTC versions of the present formulations that are more freely available.

Thus, embodiments of the present invention will provide unique benefits when compared to immediate release and/or drug combination products that are currently on the market. Such benefits include: (1) dosing two times a day, instead of every four to six hours, which improves patient compliance and that the correct dose of drug is delivered; (2) incorporating the use of pseudoephedrine into a prescription cough and cold medicine, which further ensures the involvement of a licensed medical professional and leads to more appropriate use of the pseudoephedrine, such as when administered in combination with other drugs; (3)—a liquid suspension offers more flexible dosing options than tablets, which allows for individualized patient treatment and the ability to titrate therapy based on symptoms; (4) reducing the potential for abuse and/or diversion of hydrocodone and/or pseudoephedrine, resulting from the processing of the APIs in the extended release portion of the formulations; and (5) using a unit of use (4 oz) or unit dose (5 ml to 10 ml) that will be easier to track and can also reduce the potential for diversion.

In addition, the present invention provides a novel oral liquid suspension formulation comprising an extended-release component comprising pellets, beads or particles containing one or more drugs, where the pellets, beads or particles are suspended in a syrup. The syrup may also contain one or more drugs. The oral liquid suspension provides superior stability over other liquid formulations in the art.

The invention contemplates the inclusion of a soluble, non-electrolytic component(s) within the bead during manufacture. Such a component will dissolve when water is absorbed into the bead and diffuse out of the reservoir and reduce osmotic pressure, thereby reducing swelling of the bead and loss of integrity of the bead coating. Accordingly, the invention encompasses the use of excipients that are capable of being mass transferred out of the drug-loaded bead when the bead absorbs water, thereby reducing pressure inside the bead and preventing the disruption of bead coatings and facilitating controlled release of the drug.

As such, the invention also contemplates reducing the water activity in the dispersion medium of the compositions of the invention consisting of chlorpheniramine, hydrocodone and pseudoephedrine as active ingredients by adding a high concentration of inactive component that is highly hydrated and capable of associating strongly with water and impeding the association of water with the drug complex of the dispersed phase. As such, water in the dispersion medium is not sufficiently attracted to the drug loaded bead, thereby eliminating dissolution of drug prior to administration and confining the drug in the dispersed phase of a suspension.

Accordingly, in one embodiment, the present invention encompasses thermodynamically stable liquid dosage drug suspensions of the compositions of the invention consisting of chlorpheniramine, hydrocodone and pseudoephedrine as active ingredients capable of providing sustained drug release when administered to a patient. Such liquid formulations are capable of achieving sustained release over 12, 24 hours and up to 48 hours. As such, the invention encompasses liquid dosage forms that need only be administered once or twice daily, ensuring ease of administration. It is envisaged by the invention that a soluble non-electrolytic component, having relatively low molecular weight may also be included in the drug complex. In one embodiment, the drug-ion exchange matrix-complex is a bead. It is also envisaged by the invention that the dispersed phase can optionally be membrane-coated with a porous or non-porous polymeric membrane. In one of the embodiments of the compositions of the invention consisting of chlorpheniramine, hydrocodone and pseudoephedrine as active ingredients, the dispersion medium also includes a highly hydrated excipient(s) capable of associating closely with water in the dispersion medium, thereby limiting the water activity in the dispersion medium, minimizing water attraction to the drug loaded bead and impeding the dissolution of drug prior to administration. In one such embodiment, drug release is activated following administration to a patient. Drug release is triggered when the suspension is placed in an environment, for example gastric or intestinal fluid, with high concentrations of water and small ions that possess the same charge as the drug, as the small ions swamp the diffuse double layer. In certain embodiments, the gastric fluid of the patient dilutes the dispersion medium after administration of the dosage form and the membrane becomes hydrated and more porous, allowing dissolution and dispersion of drug from the beads.

The dispersed phase comprises three drugs or active ingredients. In the liquid form controlled release compositions of the invention, the dispersed phase consists of chlorpheniramine, hydrocodone and pseudoephedrine as active ingredients. It is also envisaged that such drugs associate in a single particulate, pellet or bead. In one of the embodiments, the drugs associate with the pharmaceutically acceptable ion-exchange matrix having a surface charge opposite that of such drugs. In one embodiment, the drugs associate with the same pharmaceutically acceptable ion-exchange matrix having a surface charge opposite that of such drugs.

In one of the embodiments, the dispersed phase contains a salt form of a drug or active ingredient. In another embodiment, the dispersed phase contains a salt form of the ion-exchange matrix. In one embodiment, the dispersed phase contains pharmaceutically acceptable salt forms of drug/s and/or the ion-exchange matrix.

In one of the embodiments of the compositions of the present invention consisting of chlorpheniramine, hydrocodone and pseudoephedrine as active ingredients, the drug or drugs in the dispersed phase have a very low rate of release into the dispersion medium before its administration to a patient, i.e., less than 5% based on the total molar amount of drug in the dispersion medium and dispersed phase. The present invention contemplates liquid form controlled release compositions consisting of chlorpheniramine, hydrocodone and pseudoephedrine as active ingredients wherein the amount of the drug released from the dispersed phase into the dispersion medium before administration to a patient is less than 30%, less than 25%, less than 20%, less than 15%, less than 10%, less than 5%, less than 0.5%, or less than 0.05% based on the total molar amount of drug in the dispersion medium and dispersed phase. In such embodiments, the dispersion medium is physically and chemically stable for more than one year, more than about 2 years, more than about 3 years, or more than 4 years. In one embodiment of the compositions of the present invention consisting of chlorpheniramine, hydrocodone and pseudoephedrine as active ingredients, the dispersion medium may be substantially devoid of free drug. In yet another embodiment of the present invention, one or more of the drugs in the dispersed phase is released into the dispersion medium before its administration to a patient, e.g., about 10%, 15%, 20%, 25%, 30%, 35%, 40%, 45% or 50% of the drug is released based on the total molar amount of drug in the dispersion medium and dispersed phase.

The compositions of the present invention consisting of chlorpheniramine, hydrocodone and pseudoephedrine as active ingredients maintain stability in the presence of the drugs in the dispersion medium, i.e., drugs that are not bound to an ion-exchange matrix. Moreover, such compositions of the present invention maintain adequate release of the drugs from the dispersed phase in the presence of free drugs in the dispersion medium. In another embodiment of such compositions of the invention, the dispersion medium contains salt form drug(s).

It is envisaged that the compositions of the present invention have a storage shelf life at room temperature conditions of at least one year, about two years, and/or three, four or five years, during which time the stability and drug release profile characteristics of such compositions are maintained.

The present invention also envisages a method of preparing a liquid form controlled release drug compositions of the invention consisting of chlorpheniramine, hydrocodone and pseudoephedrine as active ingredients, wherein preparing the dispersed phase comprises blending of the active ingredients and ion exchange matrix powders. In one of the embodiments, a dispersed phase is prepared with ionic or salt forms of the active ingredients mixed with a salt form of an ion-exchange matrix.

In a specific embodiment, the salt forms of chlorpheniramine, pseudoephedrine and hydrocodone are allowed to mix with sodium alginate powders in the presence of water and one or more other ingredients. Non-limiting examples of the other ingredients include microcrystalline cellulose, forming drug-loaded beads. In a more embodiment, the drug-loaded beads further comprise lactose. In certain embodiments, the resulting beads are coated with EUDRAGIT® in the presence of triethyl citrate and talc, and cured in an oven. The coated beads are suspended in a dispersion medium that comprises salt forms of chlorpheniramine and hydrocodone, water and sucrose. In certain specific embodiments, the dispersion medium comprises Syrup NF. The dispersion medium can also further comprise preservatives and other non-active additives. In such embodiments, the resulting liquid sustained release product is capable of maintaining physical stability in a bottle and capable of achieving controlled release of drug product when administered to a patient. The dosage forms of the invention are particularly beneficial to patients who require administration of more than one drug at a time and to patients who require chronic drug administration.

In one specific embodiment, the present invention envisages treatment of cold symptoms using liquid form controlled release drug composition consisting of the following active ingredients: chlorpheniramine, hydrocodone and pseudoephedrine. In one embodiment, the present invention overcomes problems and disadvantages previously associated with formulations of immediate release (IR) and/or combination products comprising an antihistamine, an antitussive, and a decongestant. The present invention provides unique benefits as compared to immediate release and/or combination cold and allergy products that are current available. The present invention integrates the benefits of three active pharmaceutical ingredients (“APIs”), i.e., chlorpheniramine (which is an antihistamine), hydrocodone (which is an antitussive) and pseudoephedrine (which is a decongestant), into one extended release (ER) (e.g., 12 hour) composition. Previously, when used together, these APIs needed to be dosed 4-6 times daily in their immediate release (IR) forms because they are not currently available in a single extended release triple-acting combination product.

In contrast to the many products currently commercially available, the present invention provides a formulation that allows extended release (ER) (e.g., 12 hour) of all three active ingredients. The present invention provides an oral formulation that comprises a novel mixture of IR and ER forms of chlorpheniramine (an antihistamine), hydrocodone (a narcotic antitussive) and pseudoephedrine (a decongestant), in a single product. This formulation results in an ER combination product that can be dosed twice daily with the same effectiveness as previously available IR forms (which have been sold and administered either individually or via combination products). The formulation is also superior to existing single and combination ER formulations in that it (a) provides both IR and ER dosages of drugs for immediate and long term drug delivery; (b) provides bioequivalent dosages of three RLDs in a single dosage form; and (c) resists abuse and diversion of the component drugs.

### BRIEF DESCRIPTION OF THE DRAWINGS

FIG. 1(A) shows the release profile of pseudoephedrine from sustained release suspensions of Formulation X.

FIG. 1(B) shows the release profile of pseudoephedrine from coated beads.

FIG. 2(A) shows the release profile of hydrocodone from sustained release suspensions of Formulation X.

FIG. 2(B) shows the release profile of hydrocodone from coated beads.

FIG. 3(A) shows the release profile of chlorpheniramine from sustained release suspensions of Formulation X.

FIG. 3(B) shows the release profile of chlorpheniramine from coated beads.

FIG. 4(A) shows the release profile of pseudoephedrine at time=3 weeks for suspensions of the base formulations (n=3, vessel 4, 5, and 6).

FIG. 4(B) shows the release profile of pseudoephedrine (pseudoephedrine hydrochloride) at time=3 weeks for suspensions of the salt formulations (n=3, vessels 1, 2, and 3).

FIG. 5(A) shows the release profile of hydrocodone at time=3 weeks for suspensions of the base formulations (n=3, vessel 4, 5, and 6).

FIG. 5(B) shows the release profile of hydrocodone (hydrocodone bitartrate) at time=3 weeks for suspensions of the salt formulations (n=3, vessels 1, 2, and 3).

FIG. 6(A) shows the release profile of chlorpheniramine at time=3 weeks for suspensions of the base formulations (n=3, vessel 4, 5, and 6).

FIG. 6(B) shows the release profile of chlorpheniramine (chlorpheniramine maleate) at time=3 weeks for suspensions of the salt formulations (n=3, vessels 1, 2, and 3).

FIG. 7 shows release profile of hydrocodone at time=0 from a sustained release suspension containing one active ingredient, hydrocodone (10 mg/5 mL), bound to alginic acid.

### DETAILED DESCRIPTION OF THE INVENTION

As used herein, the term “patient” includes, but is not limited to any animal such as a bird (e.g., poultry) or a mammal, including humans, domestic and farm animals, and zoo, sports and pet companion animals such as household pet and other domesticated animals such as, but not limited to, cattle, sheep, ferrets, swine, horses, rabbits, goats. As used herein, the terms “subject” and “patient” can also be used interchangeably. In one embodiment, a patient is a mammal such as a non-primate (e.g., cows, pigs, horses, cats, dogs, rats, etc.) and a primate (e.g., monkey and human).

As used herein, the terms “treat” and “treatment” refer to both therapeutic treatments and prophylactic or preventative measures, wherein the object is to prevent or attenuate an undesired physiological condition, disorder or disease or obtain beneficial or desired clinical results. For purposes of this invention, beneficial or desired clinical results include but are not limited to, alleviation of symptoms; diminishment of extent of condition, disorder or disease; stabilized (i.e., not worsening) state of condition, disorder or disease; delay or slowing of condition, disorder or disease progression; amelioration of the condition, disorder or disease state; remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

As used herein, the phrase “electrolytic drug” refers to the pharmaceutically acceptable ionic form of a drug that is capable of being ionized by dissociation or protonation.

As used herein, the term “drug” means an active ingredient, e.g., a therapeutically active ingredient; and the terms “drug,” “active ingredient,” and “active pharmaceutical ingredient” (or “API”) are used interchangeably.

As used herein, the terms “extended release phase” or “extended release portion” refer to the phase or portion of a drug composition which undergoes sustained release over time upon administration of the composition to a patient; and in a liquid form controlled release composition, such terms refer to the dispersed solid phase of the composition, i.e., the dispersed phase.

As used herein, the terms “immediate release phase” or “immediate release portion” refer to the phase or portion of a drug composition which undergoes immediate release upon administration of the composition to a patient; and in a liquid form controlled release composition, such terms refer to the liquid phase of the composition, i.e., the dispersion medium.

As used herein, the term “diffusible counterion” refers to a pharmaceutically acceptable ion that is capable of displacing or replacing the electrolytic drug from the ion-exchange matrix. If the diffusible counterion has a positive charge, it is referred to herein as a diffusible counter-cation. Non-limiting examples of diffusible counter-cations such as, e.g., sodium, potassium, magnesium or calcium. If the diffusible counterion has a negative charge, it is referred to herein as a diffusible counter-anion. Non-limiting examples of diffusible counter-anions include, e.g., chloride, bromide, iodide, and phosphate.

As used herein, the term “water soluble” when used in connection with an electrolytic drug means having a solubility of greater than about 3 g of the electrolytic drug in 100 ml of water at any physiologically relevant pH. In particular embodiments, the term water soluble means having a solubility of greater than 1 g of the electrolytic drug in 100 ml of water at any physiologically relevant pH.

As used herein, the phrase “base form of the amine” when used in connection with a drug or an ion-exchange matrix means that substantially all the amine-nitrogen atoms are unprotonated and have a neutral charge.

As used herein, the phrase “acid form” when used in connection with a drug or an ion-exchange matrix means that substantially all the acid groups are in their undissociated, uncharged acid form.

As used herein, the phrase “highly hydrated” describes a component having sufficient hydrogen bonds to restrict the thermodynamic activity of water.

| Abbreviation | Definition |
|---|---|
| ACCP | American College of Chest Physicians |
| ADME | Absorbtion, Distribution, Metabolism, Excretion |
| API | Active Pharmaceutical Ingredient |
| AUC | Area under the concentration versus time curve from time |
| | 0 to infinity |
| AUCₜ | Area under the concentration versus time curve from time |
| | 0 to the last measured concentration (Cₜ) |
| BA | Bioavailability |
| BE | Bioequivalent |
| Cₘₐₓ | Maximum plasma concentration; the highest concentration |
| | observed during a dosage interval |
| Cₘᵢₙ | Minimum plasma concentration; the lowest concentration |
| | observed during a dosage interval |
| CPM | Chlorpheniramine maleate |
| CNS | Central Nervous System |
| ER | Extended Release |
| FDA | U.S. Food and Drug Administration |
| GRASE | Generally Recognized as Safe and Effective |
| HC | Hydrocodone |
| IND | Investigational New Drug |
| IR | Immediate Release |
| OTC | Over-the-counter |
| PSE | Pseudoephedrine |
| RLD | Reference Listed Drug |
| PK | Pharmacokinetic |
| SAE | Serious adverse event |
| t_{1/2} | Terminal half-life; |
| tₘₐₓ | The time that Cₘₐₓ was observed |

In one embodiment, the invention relates to liquid sustained release formulations consisting of chlorpheniramine, hydrocodone and pseudoephedrine as active ingredients and comprising drug-loaded beads produced by combining an ion-exchange matrix that is a hydrophilic colloid and the drugs having a charge opposite that of the matrix in the presence of water.

The liquid sustained release formulations of the invention consisting of chlorpheniramine, hydrocodone and pseudoephedrine as active ingredients are capable of controlled release spanning about 8 hours, about 10 hours, about 12 hours, about 16 hours, about 18 hours, about 24 hours, up to about 48 hours. In certain embodiments, from about 15%, 20%, 25%, 30%, 35%, 40%, or 45% and up to about 50%, 55%, 60%, 65%, 70%, 75%, 80%, 85%, 90%, 95% or 100% of the total molar amount of a drug in a liquid sustained release formulation of the invention is released over the time period of 12 hours, 16 hours, or 24 hours upon administration of such formulation to a patient. In certain embodiments, about 40% to about 100%, or 40% to 60%, or 45% to 55%, of the total molar amount of a drug in a liquid sustained release formulation of the invention is released over the time period of 12 hours upon administration of such formulation to a patient. In certain embodiments of the invention, from about 40% to about 100%, or 40% to 60%, or 45% to 55%, of the total molar amount of a drug in an extended release phase of a liquid sustained release formulation of the invention is released over the time period of 12 hours upon administration of such formulation to a patient. In certain embodiments, from about 40% to about 100%, or 70% to 100%, or 90% to 100%, of the total molar amount of a drug in a liquid sustained release formulation of the invention is released over the time period of 24 hours upon administration of such formulation to a patient. In certain embodiments of the compositions of the invention consisting of chlorpheniramine, hydrocodone and pseudoephedrine as active ingredients, from about 40% to about 100%, or 70% to 100%, or 90% to 100%, of the total molar amount of a drug in an extended release phase of a liquid sustained release formulation of the invention is released over the time period of 24 hours upon administration of such formulation to a patient.

In certain embodiments of the compositions of the invention consisting of chlorpheniramine, hydrocodone and pseudoephedrine as active ingredients, the liquid sustained release drug delivery systems comprise beads which contain a drug and are coated with a material that controls the release of the drug. In one embodiment, the coating is a barrier through which the drug must diffuse before it becomes bioavailable.

In other embodiments, the drug is in its salt form, e.g., a pharmaceutically acceptable salt form. Suitable pharmaceutically acceptable salts of drugs include, but are not limited to, sodium, potassium, lithium; calcium, magnesium; aluminum and zinc or other similar metals; ammonia and organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine; also sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, hydrochloride, pamoate (i.e., 1,1′-methylene-bis-(2-hydroxy-3-naphthoate), embonate, estolate, and tosylate. As such, use of salt forms of drugs provide multiple advantages. A drug substance often has certain suboptimal physicochemical or biopharmaceutical properties that can be overcome by pairing an ionized basic or acidic drug molecule with a counterion to create a salt version of the drug. In addition, pharmaceutically acceptable drugs, suitable for use in humans, are generally more easily available from manufacturers in salt forms rather than base forms. The choice of salt is governed largely by the acidity or basicity of the ionizable group, the safety of the counterion, the drug indications and the intended dosage form. One skilled in the art would know how to select a pharmaceutically acceptable salt form of a drug (see, e.g., Kumar, L., et al., “Salt Selection in Drug Development,” in Pharmaceutical Technology 3(32) (2008)).

In alternate embodiments of the compositions of the invention consisting of chlorpheniramine, hydrocodone and pseudoephedrine as active ingredients, the dispersion medium comprises a component(s) that is highly hydrated and capable of associating with water. Such components attract water from the dispersion medium such that water activity outside the bead and in the dispersion medium is less than inside the bead. In such embodiments, the dispersion medium has low enough water activity to preclude water diffusion into the drug-loaded bead and the development of internal osmotic pressure until the formulation is administered and the dispersion medium is diluted. In certain embodiments of the invention, the component is a non-electrolytic excipient such as, but not limited to, sucrose, dextrose, maltose, manitol, sorbitol, glycerin, or low molecular weight polyethylene glycol. In certain such embodiments, the drug delivery systems contemplated by the invention are activated by water (e.g., water in gastric fluid). In such embodiments, when the activity of water outside the bead in the dispersion medium is greater than the activity of water inside the bead, water will diffuse through the coating and dissolve soluble components of the bead and create a reservoir of diffusible free drug. Such free drug can permeate the coating which can control release of the drug to the patient.

In one embodiment, the composition of the invention has a shelf life of 6 months or more. In certain embodiments, the composition of the invention maintains stability prior to administration to a patient for 6 months or more.

Further, in another embodiment of the compositions of the invention consisting of chlorpheniramine, hydrocodone and pseudoephedrine as active ingredients, the dispersion medium comprises a highly hydrated excipient. Specifically, in one such embodiment, the dispersion medium comprises 50% to 70% on a weight by weight basis of a highly hydrated excipient. In yet another such embodiment, the highly hydrated excipient is sucrose.

The compositions of the invention, in one embodiment, further comprise an excipient selected from the group consisting of sweetening agents, flavoring agents, coloring agents, and any combination thereof. In another embodiment, the composition, further comprises a dispersion additive selected from the group consisting of stabilizing agents, dispersion agents, and any combination thereof.

In certain embodiment, in the compositions of the present invention, the active ingredients consist of chlorpheniramine, hydrocodone and pseudoephedrine.

In one such embodiment of the invention, the dispersed phase further comprises a pharmaceutically acceptable ion-exchange matrix and a water-soluble electrolytic drug(s) associated with the ion-exchange matrix, wherein the surface charge of the ion-exchange matrix is opposite that of the electrolytic drug. In another such embodiment of the invention, the dispersed phase is membrane-coated. In particular embodiments of the invention, the membrane is polymeric. The membrane can be porous or non-porous. In one embodiment of the invention, the membrane controls diffusion of the drug. In yet other embodiments of the invention, the dispersed phase comprises drug-loaded beads that include a low molecular weight, non-electrolytic soluble excipient(s) capable of dissolving and diffusing out of the beads when water is absorbed into the bead and reducing osmotic pressure inside the beads. In one of the embodiments of the invention, the low molecular weight excipient is lactose. In another embodiment of the invention, the dispersion medium further comprises a highly hydrated excipient that attracts water in the dispersion medium. The invention contemplates a high concentration of a highly hydrated excipient, e.g., 50% to 70%, or 55% to 70%, or 45% to 70%, or 55% to 65%, or 60% to 70% on a weight by weight basis of a highly hydrated excipient in the dispersion medium. In one of the embodiments of the invention, the highly hydrated excipient in the dispersion medium is sucrose, for example 65% sucrose on a weight by weight basis. In a certain embodiment of the invention, one or more of the drugs or active ingredients in a dispersed phase and/or dispersion medium are not in a base form. In another embodiment of the invention, one or more of the drugs or active ingredients in a dispersed phase and/or dispersion medium are in a salt form. In yet another embodiment of the invention, the dispersed phase comprises a mixture of drug and ion-exchange matrix powders, wherein the drug(s) and the ion-exchange matrix are in a salt form, e.g., a pharmaceutically acceptable salt form.

In yet another embodiment of the compositions of the invention consisting of chlorpheniramine, hydrocodone and pseudoephedrine as active ingredients, one or more drugs or active ingredients in a dispersed phase and/or dispersion medium is not in a salt form. In one such embodiment, one or more drugs or active ingredients in a dispersed phase and/or dispersion medium are in a base form.

The ion-exchange matrix can be a high molecular weight organic compound such as an oligomer, co-oligomer, polymer or co-polymer; a porous inorganic network solid such as, e.g., a zeolite; and/or combinations thereof, which have charged surfaces and are capable of retaining an oppositely-charged ion. As used herein, the phrase “cation-exchange matrix” refers to an ion exchange matrix which is capable of retaining a cationic form of a drug. As used herein, the phrase “anion-exchange matrix” refers to an ion exchange matrix which is capable of retaining an anionic form of a drug.

The design of the of the compositions of the invention consisting of chlorpheniramine, hydrocodone and pseudoephedrine as active ingredients and the selection of appropriate components are predicated on the charge of the therapeutically active ingredient (drug). The invention is suitable for the administration of drugs which are uncharged bases or acids; or cationic or anionic drugs, which are strong electrolytes as well as weakly acidic drugs above their pKa (anions) and weakly basic drugs below their pKa (cations). When the drug is an ion, the ion-exchange matrix must have a charge opposite that of the drug ion. When the drug is an uncharged base or acid, the ion-exchange matrix is in the form of an acid or base, respectively. If the electrolytic drug is a cation, then an ion-exchange matrix with a negative surface functionality must be utilized as ion-exchange matrix. The compositions of the present invention consist of chlorpheniramine, hydrocodone and pseudoephedrine as active ingredients, which are positively charged cationic drugs.

Cation- and anion-exchange matrices are well-known in the art. Non-limiting examples of useful cation-exchange matrices include cation-exchange resins such as, e.g., resins having polymer backbones comprising styrene-divinyl benzene copolymers and having pendant sulfonate groups, available from Rohm and Haas, Philadelphia, Pa., and sold under the tradename AMBERLITE™ IRP69; methacrylic acid and divinyl benzene co-polymers which have a carboxylate functionality, available from Rohm and Haas, and sold under the tradenames AMBERLITE™ IRP64 and IRP88; hydrophilic colloids such as, e.g., alginate, carboxymethylcellulose, croscarmellose, microcrystalline cellulose, xanthan gum, carboxy vinyl polymers such as carbomer 94, gelatin; or any combination thereof. In one embodiment, the cation-exchange matrix is alginate, carboxymethylcellulose, microcrystalline cellulose, xanthan gum, carboxy vinyl polymer, gelatin or any combination thereof.

Non-limiting examples of useful anion-exchange matrices include anion-exchange resins such as, e.g., resins having polymer backbones comprising styrene-divinyl benzene copolymers and having pendant ammonium or tetraalkyl ammonium functional groups, available from Rohm and Haas, Philadelphia, Pa., and sold under the tradename DUOLITE™ AP143; and hydrophilic colloids such as, but not limited to, chitosan, polylysine, or gelatin; and any combination thereof. In one embodiment, the anion-exchange matrix is chitosan, polylysine, gelatin or any combination thereof.

In certain embodiments of the compositions of the invention consisting of chlorpheniramine, hydrocodone and pseudoephedrine as active ingredients, the ion-exchange matrix is water-insoluble. In an alternate embodiment of such compositions of the invention, the ion-exchange matrix is water soluble. In such embodiments, the ion-exchange matrix is capable of being solvated with the dispersion medium, and, in one embodiment, is a hydrophilic colloid. The invention contemplates hydrophillic colloids including but not limited to natural materials such as starch, agar, cellulose, alginic acid, guar gum, xanthan gum, gelatin, acacia, and albumin have been used for applications that range from something as simple as a wet binder to something as novel as a component of microspheres. Non-limiting synthetic examples include methylcellulose, carboxymethylcellulose, hydroxypropylmethyl cellulose, methylacrylic acid, polylactic acid, polyglycolic acid, and polyanhydrides are also widely deployed in non-limiting applications ranging from the traditional, such as wet granulation, to the more contemporary, such as functional coatings and biodegradable implants.

In other embodiments, the cation-exchange matrix is a hydrophillic colloid. In such embodiments, the cation-exchange matrix is alginate, carboxymethylcellulose, microcrystalline cellulose, xanthan gum, carboxyvinyl polymers such as carbomer 94, or any combination thereof. In certain embodiments, the hydrophilic colloid is cross-linked to reduce swelling. In an embodiment, the ion-exchange material is calcium alginate. In another embodiment, the ion-exchange matrix is sodium alginate.

In other embodiments, the anion-exchange matrix is a hydrophillic colloid. In such embodiments, the anion-exchange matrix is chitosan, polylysine, gelatin, or any combination thereof. In certain embodiments, the hydrophilic colloid is cross-linked to reduce swelling.

The water soluble electrolytic drug associates with the ion-exchange matrix and forms an ion-exchange matrix drug complex.

In certain embodiments, the ion-exchange matrix drug complex is in the form of a particulate or bead. The particulate or bead is of a size which can be administered orally in a liquid dosage form in the compositions of the invention consisting of chlorpheniramine, hydrocodone and pseudoephedrine as active ingredients. In one embodiment of the invention, the particulate or bead is of a size and/or density such that it does not settle in suspension. In certain embodiments, the particulate or bead does not have undesirable patient attributes. In particular embodiments, the diameter of the particulate or bead ranges from about 0.01 μm to about 2000 μm; in another embodiment, from about 0.1 μm to about 1000 μm; and in another embodiment, from about 1 μm to about 1000 μm. In other embodiments, the diameter of the particulate or bead is greater than 2000 μm, greater than 3000 μm, or greater than 5000 μm. In alternate embodiments, the diameter of the particulate or bead is no greater than 2000 μm, no greater than 1000 μm, no greater than 500 μm, no greater than 50 μm, or no greater than 1 μm. In one embodiment, the diameter of the particulates, pellets or beads is about 600 μm. In some embodiments, the diameter of the particulates, pellets or beads is about 200 μm, about 300 μm, about 400 μm, about 500 μm, about 600 μm, about 700 μm, about 800 μm, or about 900 μm.

The core may further comprise pharmaceutically acceptable processing aid useful for forming solid dosage forms including, but limited to, bulking agents such as starch, titanium oxide, and silica; preservatives; stabilizers such as antioxidants; lubricants such as vegetable oils; and the like.

In an embodiment, the ion-exchange matrix drug complex further comprises a low molecular weight, soluble, non-electrolytic excipient. Such an excipient is capable of dissolving in water and diffusing out of the bead when the bead absorbs water and thereby reduces osmotic pressure inside the bead. The excipient must have a low enough molecular weight to permeate any membrane coating the bead. In various embodiments, the amount of excipient included in the bead can affect the rate of drug release. In various embodiments, the excipient is present in the bead at about 5% to about 10%, at about 10% to about 20%, at about 20% to about 30% at about 30% to about 40%, at about 40% to about 45%. In one embodiment, the excipient is lactose. In certain such embodiments, the more lactose incorporated in the dispersed phase during manufacturing, the faster the release of drug from the bead after administration. In various embodiments, lactose is present in the bead at about 5% to about 10%, at about 10% to about 20%, at about 20% to about 30% at about 30% to about 40%, at about 40% to about 45%. In certain embodiments, lactose is present in the bead at about 20% to about 30%. Other examples of soluble non-electrolytic excipients encompassed by the invention include but are not limited to dextrose, maltose, manitol, sorbitol, glycerin, or low molecular weight polyethylene glycol.

In one embodiment, the ion-exchange matrix drug complex further comprises a diffusion-controlling membrane coating. The membrane coating is useful for further controlling diffusion of counterions into and drug out of the ion-exchange matrix. Thus, the diffusion-controlling membrane coating is useful for controlling the release of the electrolytic drug into the dispersion medium and/or the digestive tract after administration to a patient. The invention encompasses the use of any membrane-coating that provides diffusion control. The coating materials may be any of a large number of natural or synthetic film-formers used alone, in admixture with each other, and in admixture with other components such as plasticizers, pigments, and other substances. In certain embodiments, the components of the coating are insoluble in, and permeable to, water. Incorporation of a water-soluble substance can be useful in altering the permeability of the coating. Diffusion-controlling membranes are known in the art. Non-limiting examples include ethylcelluloses such as SURELEASE® (Colorcon, Westpoint, Pa.); methylmethacrylate polymers such as EUDRAGIT® (Röhm Pharma, GmbH, Weiterstat, Germany); cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. In an embodiment, the coating is a methylmethacrylate polymer.

In one embodiment, the diffusion-controlling membrane is selected from the group consisting of ethylcellulose, methylmethacrylate, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, and combinations thereof. In one embodiment, the diffusion-controlling membrane is ethylcellulose, methylmethacrylate, or combinations thereof. In another embodiment, the diffusion-controlling membrane coating is from about 20% to about 30% by weight based on the total weight of the coating and the ion-exchange matrix drug complex.

In one embodiment, the ion-exchange matrix drug complex is coated with from about 1% up to about 75% of diffusion-controlling membrane based on the total weight of the ion-exchange matrix drug complex and the diffusion-controlling membrane; in another embodiment, from 5% to about 50%; and in one embodiment, from about 10% to about 30%, and in another embodiment from about 20% to about 25%. Typically, the more coating, the more delay in the release of the drug.

In one embodiment, drug-loaded alginate beads are coated with sufficient EUDRAGIT® (Rohm) RS 30 D to provide a coated bead having from about 20% to about 30% by weight of coating based on the total weight of the coating and the drug-loaded alginate beads.

In another embodiment, the diffusion-controlling membrane coating of the ion-exchange matrix drug complex further comprises a plasticizer. Plasticizers are useful to increase flexibility and reduce brittleness of the coating. Plasticizers also affect drug release rate. A plasticizer lowers the glass transition temperature of the coating and that facilitates coalescence of the applied droplets into a coherent film, and affects permeability of the coating. Plasticizers are known in the art. Non-limiting examples of plasticizers include triethyl citrate, diethyl sebacate, diethyl phthalate, tributyl citrate, and acetyl tributyl citrate. In one embodiment, the plasticizer is triethyl citrate. In one embodiment, the ion-exchange matrix drug complex contains from about 0.1% up to 30%, or from about 0.5% up to about 20%, from about 1% to about 20%, or from about 2% to about 10%, of the plasticizer based on the total weight of the ion-exchange matrix drug complex and the diffusion-controlling membrane.

The composition of the present invention consisting of chlorpheniramine, hydrocodone and pseudoephedrine as active ingredients is stable in the presence of ionic components. In one embodiment, such composition of the present invention is stable in the presence of ionic components in the dispersion medium. In such embodiment, the composition of the present invention is stable in the presence of diffusible counterions in the dispersion medium. In another such embodiment, the composition of the present invention is stable in the presence of electrolytic drugs, i.e., chlorpheniramine, hydrocodone and pseudoephedrine, in the dispersion medium. The composition of the present invention consisting of chlorpheniramine, hydrocodone and pseudoephedrine as active ingredients maintains stability in the presence of such drugs in a free form in the dispersion medium, i.e., drugs that are not bound to an ion-exchange matrix. The composition of the present invention maintains adequate sustained release profile of the drugs from the dispersed phase in the presence of free drugs in the dispersion medium. In another embodiment of the invention, the dispersion medium consists of chlorpheniramine, hydrocodone and optionally pseudoephedrine as active ingredients in the immediate release form. In such embodiments, the drugs in the dispersion medium are not bound to an ion-exchange matrix. In one of such embodiment, the dispersion medium contains a salt form of a drug(s).

In one embodiment, the liquid form controlled release compositions of the invention consisting of chlorpheniramine, hydrocodone and pseudoephedrine as active ingredients are stable in the presence of free drugs and/or diffusible counterions in the dispersion medium. In one such embodiment, the composition of the invention comprises an extended release phase and an immediate release phase, wherein the immediate phase contains a certain amount of free drug, wherein the amount of the drug released from the extended release phase into the immediate release phase before administration to a patient is less than 30%, or less than 25%, or less than 20%, or less than 10%, less than 5%, less than 0.5%, or less than 0.05% based on the total molar amount of drug in the dispersion medium and dispersed phase.

The present invention is directed to the liquid form controlled release compositions that contain three drugs used for three different therapeutic indications, e.g., chloropheniramine for the treatment of allergies and rhinorrhea, hydrocodone for the treatment of cough, and pseudoephedrine for the treatment of nasal obstruction.

In yet another embodiment of the liquid form controlled release composition of the invention consisting of chlorpheniramine, hydrocodone and pseudoephedrine as active ingredients, one or more drugs in the dispersed phase leach into the dispersion medium before its administration to a patient. In such embodiment, about 10%, 15%, 20%, 25%, 30%, 35%, 40%, 45%, 50%, 55%, 60%, 70% and up to 75% of the drug is released from the dispersed phase into the dispersion medium before its administration to a patient, based on the total molar amount of drug in the dispersion medium and dispersed phase. In one such embodiment, from about 15% to about 35%, and another embodiment about 25% of the drug is released from the dispersed phase into the dispersion medium before its administration to a patient, based on the total molar amount of drug in the dispersion medium and dispersed phase.

In certain embodiments, the weight ratio of a drug in an immediate release phase to the same drug in an extended release phase of the liquid sustained release compositions of the invention consisting of chlorpheniramine, hydrocodone and pseudoephedrine as active ingredients (“IR/ER ratio”) is about 0:100, or about 5:100, or about 10:100, or about 15:85, or about 20:80, or about 25:75, or about 30:70, or about 35:65, or about 40:60, or about 45:50 or about 50:50. In one such embodiment, IR/ER ratio of a drug is about 25:75. In one specific embodiment of the present invention the weight ratio of chlorpheniramine in the immediate release portion to the extended release portion of the oral composition of the invention is about 25:75, and the weight ratio of hydrocodone in the immediate release portion to the extended release portion is about 25:75, and the weight ratio of pseudoephedrine in the immediate release portion to the extended release portion is from about 25:75 to about 0:100.

In other embodiments of the compositions of the invention consisting of chlorpheniramine, hydrocodone and pseudoephedrine as active ingredients, the drug composition contains from 0.1-0.5, 0.5-1 mg, 1-5 mg, 5-10 mg, 10-15 mg, 15-20 mg, 20-25 mg, 25-30 mg, 30-40 mg, 40-50 mg, 50-60 mg, 60-70 mg, 70-80 mg, 80-90 mg, 90-100 mg, 100-120 mg, 120-140 mg, 140-160 mg, 160-180 mg, 180-200 mg, 200-220 mg, 220-240 mg, 240-260 mg, 260-280 mg, 280-300 mg, 300-350 mg, 350-400 mg, 400-450 mg, 450-500 mg, up to 600 mg, up to 700 mg, up to 800 mg, up to 900 mg, up to 1000 mg of each of the drug/s or active ingredient/s per 1 ml of the single dose of the liquid form controlled release drug composition. In yet another embodiment of the invention, such drug composition contains from 0.1-0.5, 0.5-1 mg, 1-5 mg, 5-10 mg, 10-15 mg, 15-20 mg, 20-25 mg, 25-30 mg, 30-40 mg, 40-50 mg, 50-60 mg, 60-70 mg, 70-80 mg, 80-90 mg, 90-100 mg, 100-120 mg, 120-140 mg, 140-160 mg, 160-180 mg, 180-200 mg, 200-220 mg, 220-240 mg, 240-260 mg, 260-280 mg, 280-300 mg, 300-350 mg, 350-400 mg, 400-450 mg, 450-500 mg, up to 600 mg, up to 700 mg, up to 800 mg, up to 900 mg, up to 1000 mg of each of the drug/s or active ingredient/s per 5 ml of the single dose of the liquid form controlled release drug composition. In some specific embodiments of the invention, such drug composition contains 1-5 mg, 5-10 mg, 10-15 mg, 15-20 mg, 20-25 mg, 25-30 mg, 30-40 mg, 40-50 mg, and up to 100-120 mg, 120-140 mg, 140-160 mg, 160-180 mg, 180-200 mg, 200-220 mg, 220-240 mg, 240-260 mg, 260-280 mg, 280-300 mg of each of the drug/s or active ingredient/s per 5 ml of the single dose of the liquid form controlled release drug composition.

In certain embodiments of the compositions of the invention consisting of chlorpheniramine, hydrocodone and pseudoephedrine as active ingredients, the dispersion medium further comprises a high concentration of excipient(s) that are highly hydrated, capable of associating with the water in the dispersion medium. Although the drugs of the invention are highly soluble in aqueous dispersion media, the presence of the highly hydrated component in the dispersion medium attracts the water in the dispersion medium necessary to begin the dissolution of drug from the drug-ion-exchange matrix complex. Only until the formulation is administered and gastric liquids (largely water) dilute the dispersion medium will the drugs dissolve and become available and begin to permeate the membrane and/or diffuse from the bead. In such embodiments, the dispersion medium is substantially devoid of free drug, for example, less than 0.5% drug or less than 0.05% drug, is in the dispersion medium. In various embodiments of the invention, the highly hydrated component is present in the dispersion medium, on a weight to weight basis, at about 10% to about 20%, at about 20% to about 30%, at about 30% to about 40%, at about 40% to about 50%, at about 50% to about 60%. In certain embodiments, the component is present at about 60% to about 65%, up to about 70%. In other embodiments of the invention, the dispersion medium comprises sucrose, or other sugar molecules. In such embodiments, the dispersion medium comprises, on a weight to weight basis, more than 10% sucrose, more than 20% sucrose, more than 30% sucrose, more than 40% sucrose, or more than 50% sucrose. In certain embodiments of the compositions of the invention consisting of chlorpheniramine, hydrocodone and pseudoephedrine as active ingredients, the dispersion medium comprises about 65% sucrose (i.e., Syrup NF), but no more than about 70% sucrose. Other examples of excipients encompassed by the invention include but are not limited to dextrose, manitol, fructose, polyethylene glycol, glycols, and glycerins. In certain embodiments of the invention, the dispersion medium comprises, on a weight to weight basis, more than 10% of dextrose, manitol, fructose, polyethylene glycol, glycol or glycerin, more than 20% dextrose, manitol, fructose, polyethylene glycol, glycol or glycerin, more than 30% dextrose, manitol, fructose, polyethylene glycol, glycol or glycerin, more than 40% dextrose, manitol, fructose, polyethylene glycol, glycol or glycerin, or more than 50% of dextrose, manitol, fructose, polyethylene glycol, glycol or glycerin. In a one embodiment of the invention, the dispersion medium comprises about 65% dextrose, manitol, fructose, polyethylene glycol, glycol or glycerin, but no more than about 70% of dextrose, manitol, fructose, polyethylene glycol, glycol or glycerin. One of skill in the art could readily determine other highly hydrated excipients that would function similarly.

The liquid form controlled release drug composition of the invention consisting of chlorpheniramine, hydrocodone and pseudoephedrine as active ingredients can further comprise a dispersion additive selected from the group consisting of stabilizing agents, dispersing agents, and the like, provided the excipients do not adversely affect the intended operation of the invention.

The liquid form controlled release drug composition of the invention consisting of chlorpheniramine, hydrocodone and pseudoephedrine as active ingredients can further comprise excipients useful in oral liquid dose formulations such as, e.g., sweetening agents, flavoring agents, coloring agents, thickeners, and the like, provided the excipients do not adversely affect the intended operation of the invention.

Advantages of the dosage form of the present invention consisting of chlorpheniramine, hydrocodone and pseudoephedrine as active ingredients is that the ion-trapping, osmotic control, and thermodynamic balancing mechanisms are generally applicable and inherently stable and the fact that effects can be implemented through the use of traditional and widely accepted pharmaceutical excipients one skilled in the art could utilize.

The cationic active agents useful in the present invention are chlorpheniramine, hydrocodone or pseudoephedrine.

In an embodiment, useful drugs include salt forms of the above mentioned electrolytic drugs. In certain embodiments, a salt form of the drug may be maleate, hydrochloride or bitartrate.

In certain embodiments, useful drugs also include the neutral forms of the above mentioned electrolytic drugs which form ions upon association or reaction with the ion-exchange matrix.

In various embodiments of the compositions of the invention consisting of chlorpheniramine, hydrocodone and pseudoephedrine as active ingredients, the ion-exchange matrix drug complex comprises ion-exchange matrix in an amount sufficient to convert the drug into ionic form. Optionally, the ion-exchange matrix drug complex comprises ion-exchange matrix in an amount more than sufficient to convert the drug into ionic form.

In one embodiment, the drugs or active ingredients associate in single particulate, pellet or bead in a liquid form controlled release drug composition of the invention consisting of chlorpheniramine, hydrocodone and pseudoephedrine as active ingredients. In one such embodiment, such drugs associate with the pharmaceutically acceptable ion-exchange matrix having a surface charge opposite that of the drugs. In an embodiment of the invention, such drugs associate with the same pharmaceutically acceptable ion-exchange matrix having a surface charge opposite that of the drugs. Binding such drugs to the same ion-exchange matrix does not interfere with the controlled release of each drug in the composition, and provides an adequate rate of release of each drug. It is envisioned that chlorpheniramine, hydrocodone and pseudoephedrine associate with an ion-exchange matrix with a negative surface functionality. In another embodiment of the invention, the dispersed phase contains pharmaceutically acceptable salt forms of the drugs or active ingredients. In yet another embodiment of the invention, the dispersed phase contains a pharmaceutically acceptable salt form of an ion-exchange matrix.

In certain embodiments, the compositions of the invention consist of chlorpheniramine, hydrocodone and pseudoephedrine as active ingredients in a single particulate, pellet or bed wherein there are no chemical or physical interactions between the drugs in such particulate, pellet or bead, and acceptable stability characteristics and an adequate drug delivery profile are achieved with a single drug release rate-controlling coating.

If each of such drugs of the invention are placed in separate beads, which are then mixed, it is possible that the mixture will not be perfectly homogenous, which will result in incorrect relative doses for the drugs. Non-homogeneity can occur either due to random fluctuations or due to different physical properties of the two or more beads in a mixture, such as a difference in weight and density. However, the above-described technology, wherein chlorpheniramine, hydrocodone and pseudoephedrine associate in a single particulate, pellet or bead, advantageously ensures homogeneity of the drug mixture and resulting dose uniformity of the respective drugs in the combination formulation, such that a given patient will receive the same amount of each of the drugs. Further, binding of such drugs to the same ion exchange matrix provides an advantage of having only one drug-bound resin complex in the dispersed phase. Furthermore, such combining of chlorpheniramine, hydrocodone and pseudoephedrine into single particulate, pellet, or bead, may reduce the surface area of particulates, pellets, or beads present in an overall drug combination product, and may increase stability of the product and active ingredients.

Another advantage of the above-described technology of the present invention is that it achieves similar or the same release profile for all drugs bound to the same resin, and does not require different doses or frequency of dosing for each drug of the invention. If different resins are used, then the release profile of each drug may be affected by differences in a patient's diet or physiology, such that a given patient may receive too much of one drug, but too little of another. By contrast, advantageously, the above-described technology of the present invention allows to achieve bioequivalence for chlorpheniramine, hydrocodone and pseudoephedrine at the same time. Also, placing such drugs in a single bead with a single release technology, allows to achieve a drug release profile that will be more consistent across the patient population.

Yet another advantage of the above-described technology, chlorpheniramine, hydrocodone and pseudoephedrine associate in a single particulate, pellet or bead, is that such technology makes it difficult to extract or separate out individual active ingredients of such drug combination. Specifically, binding of such active ingredients to the same ion-exchange matrix makes extraction or separation of any single active ingredient exceedingly difficult for an untrained individual. For example, if such drugs are bound to one ion-exchange matrix, it is not possible to partially isolate one drug from others on the basis of differences in the physical properties, such as densities, of individual one drug/one ion-exchange matrix complexes. Because of difficulty of isolation of any single active ingredient from products produced using the above-described technology of the present invention, such products will have a decreased potential for abuse or illegal use of any single ingredient in the product. Thus, the technology of the present invention enables manufacture of combination drug products that include chlorpheniramine, hydrocodone and pseudoephedrine such that the resulting product has a decreased potential for drug abuse and diversion.

In yet another embodiment, chlorpheniramine, hydrocodone and pseudoephedrine associate with different pharmaceutically acceptable ion-exchange matrices having a surface charge opposite that of the respective drugs.

In an embodiment, the compositions of the invention are stable for a long period of time, i.e., for at least 1 month, for at least 3 months, for at least 6 months, for one year, for two years, or for three, four, five or more years. Specifically, the compositions of the invention maintain chemical, physical and microbiological stability for above-indicated periods of time. One of skill in the art would know how to assess chemical, physical and microbiological stability of a drug composition. Stability characteristics of a drug composition, e.g., physical, chemical and microbiological stability characteristics, determine how long a drug or an active ingredient can be stored in a bottle in a final composition ready for administration to a patient. In one of the embodiments, the compositions of the invention are stable at room temperature for at least 1 month, for at least 3 months, for at least 6 months, for one year, for two years, or for three, four, five or more years. In such embodiments, the compositions of the present invention possess chemical, physical and microbiological stability for at least 1 month, for at least 3 months, for at least 6 months, one year, two years, or three, four, five or more years.

Chemical stability is manifested in structural integrity of drugs or active ingredients in the composition over time. Chemical stability may be assessed using chromatographic assays and/or potency measurements. Such assays detect the presence of a drug in a composition and presence or absence of degradation products. The drug is considered chemically stable at a time X, wherein X is a longer time period than zero (i.e., the time when a composition is manufactured), if at that time X 90-100% of the drug, which was present at time zero, is present and demonstrates adequate structural integrity characteristics, or the same or similar, or not significantly different structural characteristics in comparison to those expected at time zero. In one embodiment, the compositions of the present invention possess chemical stability for at least 1 month, for at least 3 months, for at least 6 months, one year, two years, or three, four, five or more years.

Physical stability of the compositions of the present invention is manifested in, e.g., integrity of a diffusion controlling membrane or a functional coating enveloping the dispersed phase, and its permeability. Physical stability may be assessed using a dissolution assay. A dissolution assay may be performed starting at a time zero (i.e., the time when a composition is manufactured) or at a time X, wherein X is any time above zero, such as, 1 week, 2 weeks, 3 weeks, 1 month, 3 months, 6 months, 1 year, 2 years, 3 years, 4 years, or 5 years. The dissolution testing shows the amount of drug released once the composition is placed in a chemical environment which is equivalent to the environment in a patient's gastrointestinal tract. The dissolution testing reflects the rate of release of a drug upon its administration to a patient. The dissolution or release profile, i.e. the amount of drug released over time at each time point measured (after its placing in an appropriate chemical environment or its administration to a patient) and the rate of release of a drug, is indicative of the physical stability of a drug composition. A physically stable drug composition at a certain time X would manifest the same, similar, or at least not significantly different dissolution or release profile assessed using a dissolution assay as would be expected from a drug composition tested at a time zero, i.e., when the assay is performed immediately after the drug is manufactured. Also, a physically stable drug composition would have a certain expected amount of a drug released at the first time point of the assay (i.e., at the start of the assay). More specifically, at the first time point of the assay, a physically stable composition, which does not have an immediate release portion, would exhibit no drug release, or very low level of drug release, i.e., a release of less than 10%, or less than 5%, or less than 1% of the drug from the extended release phase. Further, a physically stable drug composition would have a certain expected rate of drug release, i.e., certain amount of drug released at each subsequent time point upon placing it in an appropriate chemical environment or its administration to a patient. In one embodiment, the compositions of the present invention possess physical stability for at least 1 month, for at least 3 months, for at least 6 months, one year, two years, or three, four, five or more years. In such embodiment, a drug composition of the invention may be stored in a bottle for at least 1 month, for at least 3 months, for at least 6 months, one year, two years, or three, four, five or more year, while maintaining its physical stability.

In certain embodiments, the compositions of the present invention also maintain microbiological stability over time. Microbiological stability of a drug composition reflects absence of contamination with microorganisms of such composition. In one embodiment, the compositions of the present invention possess microbiological stability for at least 1 month, for at least 3 months, for at least 6 months, one year, two years, or three, four, five or more years.

Due to the stability characteristics of the liquid form controlled release compositions of the present invention, such compositions have a long shelf life, i.e., shelf life of one year or more. It is envisaged that the compositions of the present invention have a storage shelf life at room temperature conditions of at least 1 month, of at least 3 months, of at least 6 months, about one year, about two years, or three, four, five or more years, during which time the stability and drug release profile characteristics of such formulations are maintained. In other embodiments, the sustained release formulation is physically and chemically stable for more than 1 month, more than 3 months, more than 6 months, more than 1 year, more than about 2 years, more than about 3 years, or more than 4 years and more than 5 years.

In another embodiment, the present invention provides a combination formulation consisting of chlorpheniramine, hydrocodone and pseudoephedrine as active ingredients, and comprising an extended release and an immediate release portion which, when administered to a patient, achieves bioequivalence to immediate release product/s containing these drug/s. One advantage is that the formulations of the present invention may achieve bioequivalence for two or more of these drugs at the same time. In another embodiment, the present invention provides a non-liquid combination formulation comprising an antitussive, an antihistamine and a decongestant, and comprising an extended release and an immediate release portion which, when administered to a patient, achieves bioequivalence to immediate release product/s containing these drug/s.

In certain embodiment, the present invention relates to a non-liquid oral extended release drug composition comprising a first portion and a second portion, wherein

the first portion comprises an antihistamine, an antitussive, and optionally a decongestant as active ingredients in an immediate release form,

the second portion comprises a particulate, pellet, or bead that comprises the antihistamine, the antitussive, and the decongestant as three active ingredients in an extended release form,

administration of a single dose of the non-liquid oral drug composition to a patient provides serum levels of the three active ingredients over a time period of at least 8 hours that are bioequivalent to serum levels achieved upon administration of an appropriate number of doses, over the same time period, of FDA-approved immediate release reference listed drug (IR RLD) compositions comprised of the active ingredients, and

the appropriate number of doses corresponds to a number of doses recommended in one or more FDA-approved labels for administration of the IR RLD compositions over the same time period.

Further, in one such embodiment, the antitussive is a narcotic antitussive. In another such embodiment, the first portion does not comprise the decongestant. In one such embodiment, the particulate, pellet or bead further comprises a coating. In another such embodiment, the antihistamine, the antitussive and the decongestant associate in a single particulate, pellet or bead. In yet another such embodiment, the particulate, pellet or bead further comprises a pharmaceutically acceptable ion-exchange matrix, wherein the antihistamine, the antitussive and the decongestant associate with the ion-exchange matrix. One such embodiment further comprises an excipient selected from the group consisting of sweetening agents, flavoring agents, coloring agents, and any combination thereof. Another such embodiment is further comprising an additive selected from the group consisting of stabilizing agents, dispersion agents, and any combination thereof.

In yet another embodiment, the present invention relates to a non-liquid oral extended release drug composition comprising a first portion and a second portion, wherein

the first portion comprises an antihistamine, an antitussive, and optionally a decongestant, as active ingredients in an immediate release form,

the second portion is a particulate, pellet or bead that comprises the antihistamine, the antitussive, and the decongestant as active ingredients in an extended release form,

administration of a sufficient number of doses of the non-liquid drug composition to a patient to achieve steady-state serum levels of the three active ingredients over a time period of greater than 24 hours yields serum levels of the active ingredients that are bioequivalent to serum levels achieved upon administration of an appropriate number of doses, over the same time period, of one or more FDA-approved immediate release drug compositions comprised of the active ingredients, and

the appropriate number of doses corresponds to a number of doses recommended in one or more FDA-approved labels for the administration of the one or more FDA-approved immediate release drug compositions over the same time period.

In certain embodiments, the present invention relates to a method for achieving in a mammal serum levels of an antihistamine, an antitussive and a decongestant over a time period of at least 8 hours that are bioequivalent to serum levels achieved upon administration of an appropriate number of doses over the same time period of FDA-approved immediate release reference listed drug (IR RLD) compositions to the same mammal, wherein the method comprises:

(a) administering to the mammal a non-liquid oral extended release drug composition comprising a first portion and a second portion, wherein the first portion comprises the antihistamine, the antitussive and optionally the decongestant as active ingredients in an immediate release form, and wherein the second portion comprises a particulate, pellet, or bead that comprises the antihistamine, antitussive and the decongestant as active ingredients in an extended release form, and

(b) achieving serum levels of the three active ingredients over a time period of at least 8 hours that are bioequivalent to serum levels achieved upon administration of an appropriate number of doses over the same time period of FDA-approved IR RLD compositions comprising the active ingredients, wherein the appropriate number of doses corresponds to a number of doses recommended in one or more FDA-approved labels for the administration of the IR RLD compositions over the same time period.

In other embodiments, the present invention relates to a method for achieving in a mammal steady-state serum levels of an antihistamine, an antitussive and a decongestant upon administration of a non-liquid oral extended release (ER) drug composition, wherein the serum levels are bioequivalent to serum levels achieved upon administration of one or more immediate release (IR) compositions comprising active ingredients and inactive ingredients, wherein said active ingredients consist of an antihistamine (e.g., chlorpheniramine), an antitussive (e.g., hydrocodone) and pseudoephedrine (e.g., pseudoephedrine) to the same mammal, wherein the method comprises:

administering to the mammal a non-liquid oral ER drug composition comprising a first portion and a second portion, wherein the first portion comprises the antihistamine, the antitussive and the decongestant as active ingredients in an immediate release form, and wherein the second portion comprises a particulate, pellet or bead that comprises the antihistamine, the antitussive and the decongestant as active ingredients in an extended release form, and

wherein administration of a sufficient number of doses of the non-liquid oral ER drug composition to the mammal to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release (IR) drug compositions comprising the active ingredients,

wherein the appropriate number of doses of the one or more FDA-approved IR drug compositions corresponds to a number of doses recommended in one or more FDA-approved labels for the administration of the one or more FDA-approved IR drug compositions over the same time period, and

wherein the appropriate number of doses of the one or more FDA-approved IR drug compositions is greater than the sufficient number of doses of the oral ER drug composition.

In one embodiment, the present invention relates to a novel formulation comprising, as active pharmaceutical ingredients (APIs), an antihistamine, an antitussive, and a decongestant, where the formulation exhibits extended release (ER) release of all three drugs. For example, the present invention provides a formulation comprising a novel mixture of immediate release (IR) and ER forms of chlorpheniramine, pseudoephedrine and hydrocodone within a single product. Novel formulations of the present invention include those that result in an IR/ER combination products that can be dosed twice daily with the same effectiveness as previously available IR products comprising all three drugs, or a combination of IR and/or ER products comprising only one or two of the drugs.

An antihistamine inhibits the release or action of histamine in the body, for example by acting as an antihistamine antagonist or inverse agonist at a relevant cell receptor, such as the H₁ receptor. Histamine causes congestion, sneezing, runny and stuffy nose, itching and watery eyes associated with allergies, colds and the flu (influenza). Antihistamines can prevent histamines from attaching to cells and causing such symptoms. Examples of antihistamines include chlorpheniramine, brompheniramine, dimenhydrinate, diphenhydramine, loratadine, meclizine and quetiapine. In one embodiment of the present invention, the antihistamine is chlorpheniramine. The term “chlorpheniramine” encompasses any form of the drug, and in one specific embodiment, chlorpheniramine is chlorpheniramine maleate (CPM), also known by the chemical name 2-pyridinepropanamine, -(4-chlorophenyl)-N,N-dimethyl-, (Z)-2-butenedioate (1:1).

Decongestants also can help relieve stuffy nose and congestion caused by a cold or the flu, sinusitis or allergies. Congestion in the nose, sinuses, and chest is due to swollen, expanded, or dilated blood vessels in the membranes of the nose and air passages. These membranes have an abundant supply of blood vessels with a great capacity for expansion (swelling and congestion). Histamine stimulates these blood vessels to expand. Decongestants, by contrast, cause constriction or tightening of the blood vessels in those membranes, which forces much of the blood out of the membranes so that they shrink, and the air passages open up again. Generally, decongestants are chemically related to adrenalin, the natural decongestant, which is also a type of stimulant. The most common oral decongestants are pseudoephedrine and phenylephrine. In one embodiment of the present invention, the decongestant is pseudoephedrine (PSE). The term “pseudoephedrine” encompasses any form of the drug, and in one specific embodiment, pseudoephedrine is pseudoephedrine hydrochloride, also known by the chemical name benzenemethanol,-[1-(methylamino)ethyl]-,[S—(R*,R*)]-, hydrochloride.

Cough medicines are generally grouped into two types: antitussives and expectorants. An antitussive is a medicine used to suppress or relieve coughing, and includes non-narcotic and narcotic antitussives. Benzonatate, dextromethorphan, carbetapentane are examples of non-narcotic antitussives. Dextromethorphan (an antitussive) and guaifenesin (an expectorant) are sometimes combined with each other. One example of a narcotic antitussive is hydrocodone (HC), also an analgesic, which is a semi-synthetic opioid derived from two of naturally occurring opiates, codeine and thebaine. The term “hydrocodone” encompasses any form of the drug. In one embodiment of the present invention, the antitussive is hydrocodone bitartrate, also known by the chemical name morphinan-6-one, 4,5-epoxy-3-methoxy-17-methyl-,(5)-, [R—(R*,R*)]-2,3-dihydroxybutane-dioate (1:1), hydrate (2:5).

Combinations of antihistamines with decongestants are currently commercially available, such as Actifed®, Allegra-D®, Chlor-Trimeton D®, Claritin D®, Contac®, Co-Pyronil 2®, Deconamine®, Demazin®, Dimetapp®, Drixoral®, Isoclor®, Nolamine®, Novafed A®, Ornade®, Sudafed Plus®, Tavist D®, Triaminic®, and Trinalin®. Antitussives are also available in combination with other drugs, such as pain relievers or antihistamines. Such combination products, known as multisymptom cold medicines, treat many symptoms at once.

As discussed on the FDA's website, however, FDA-approved IR hydrocodone antitussive formulations contain only hydrocodone bitartrate and homatropine methylbromide, such as in Hycodan®, Mycodone®, and Tussigon®. Only two ER antitussive formulations containing hydrocodone and chlorpheniramine are currently approved, Tussionex Pennkinetic® (suspension) and TussiCaps® (capsule). See www.fda.gov/CDER/drug/unapproved_drugs/hydrocodone_qa.htm; accessed Apr. 11, 2008. Notably, cough suppressants that combine hydrocodone and homatropine with other drugs, like an expectorant such as guaifenesin, or a decongestant such as phenylephrine or pseudoephedrine, are currently unapproved in any form. Thus, no FDA-approved drug comprising hydrocodone and a decongestant, such as PSE, is available.

Consequently, as mentioned above, the present invention differs from previously available combination products because the novel formulations described herein comprise three APIs, i.e., an antihistamine, an antitussive, and a decongestant, and exhibit ER release of all three APIs in the body via a single oral product. In one embodiment, a novel formulation is dosed once every 12 hours. Other embodiments include those dosed every 8 hours, 16 hours, 24 hours, etc.

In one embodiment, the product is a liquid dispersion of ER coated pellets in a syrup intended for the treatment of cough, cold, and allergy symptoms. For example, a formulation may contain 10 or 15 mg hydrocodone bitartrate, 120 mg pseudoephedrine hydrochloride and 8 mg chlorpheniramine maleate in combination per adult dosage (5 ml). Salt forms of the drugs may be used, but other forms of the drugs, including the base forms, may also be used. These active ingredients have extensive human experience dosed either individually or in combination as both prescription and over-the-counter (OTC) cough cold medications.

In one embodiment of the present invention, the ER portion of the formulation corresponds to coated beads, particulates or pellets within a liquid suspension, with an IR portion located in the liquid suspension. In one embodiment, formulations of the present invention are prepared using technology described in published patent applications owned by UPM Pharmaceuticals (see U.S. Ser. No. 10/724,276, filed on Nov. 26, 2003, U.S. Ser. No. 11/150,572 filed on Jun. 9, 2005, and U.S. Ser. No. 11/198,937 filed on Aug. 4, 2005), hereby incorporated by reference, which relate to the production and use of a certain type of ER beads in suspension.

Alternatively, the ER portion in the present invention may comprise a solid dosage form such as capsule, tablet, or other oral solid, with an IR portion as a secondary layer or medium outside the ER portion. In certain embodiments, oral solid formulations contain no liquid components, i.e., such formulations do not contain a liquid phase or a dispersion medium. In such embodiments, the IR portion of the oral solid formulations does not comprise a liquid phase or a dispersion medium. In such embodiments, the oral solid formulation is not mixed with a liquid phase, e.g., a dispersion medium, prior to administration to a subject. Likewise, in one embodiment, a single combination ER product exhibits a specific IR to ER ratio, where the ER component is in a particulate, pellet, or bead and the IR portion is outside (e.g., suspended in syrup; in powder in a capsule, tablet, etc.). The ratio achieves blood serum levels that are bioequivalent (BE) to reference listed drugs (RLDs) at single-dose and steady-state conditions.

In certain embodiments, the invention relates to an extended release drug composition in a non-liquid form. A “non-liquid form” as used herein refers to a composition that is not a liquid form composition comprising a dispersed phase, which comprises an ion-exchange matrix drug complex, and a dispersion medium, as described in U.S. Ser. No. 10/724,276, filed on Nov. 26, 2003, U.S. Ser. No. 11/150,572 filed on Jun. 9, 2005, and U.S. Ser. No. 11/198,937 filed on Aug. 4, 2005).

In other embodiments, administration of the present formulations achieves certain specific blood serum ranges (as measured by AUC, Tₘₐₓ, T_{1/2}, etc.) in humans over time, where the levels are safe and effective for ER 12-hour release and BE to immediate release RLDs at both single-dose and steady-state conditions.

In one embodiment, an oral extended release drug composition comprises a first portion and a second portion, wherein the first portion comprises an antihistamine, an antitussive, and optionally a decongestant, as active ingredients in an immediate release form, and wherein the second portion comprises particulates, pellets, or beads wherein each particulates, pellets, or beads comprises the same antihistamine, antitussive and decongestant as active ingredients in an extended release form. In another embodiment, administration of a single dose of this drug composition to a patient provides serum levels of the three active ingredients over a time period of at least 8 hours, such as 8, 12, 18 or 24 hours, that are bioequivalent to serum levels achieved upon administration of an appropriate number of doses over the same time period of FDA-approved IR reference listed drug (RLD) compositions comprising the active ingredients, wherein the appropriate number of doses corresponds to a number of doses recommended in FDA-approved labels for the administration of the FDA-approved IR drug compositions over the same time period.

In certain embodiments, the extended release portion is in the form of a particulate, pellet or bead. The particulate, pellet or bead is of a size which can be administered orally in a liquid or solid dosage form. In one embodiment, the particulate, pellet or bead is of a size and/or density such that it does not settle in suspension. In some embodiments, the diameter of the particulate, pellet or bead ranges from about 0.01 μm to about 2000 μm; in another embodiment, from about 0.1 μm to about 1000 μm; and in another embodiment, from about 1 μm to about 1000 μm. In one embodiment, the diameter of the particulates, pellets or beads are about 600 μm.

In certain embodiments, a suspension formulation comprising an antihistamine, an antitussive and pseudoephedrine, wherein the formulation exhibits IR and ER release of all three drugs, takes advantage of the fact that pseudoephedrine releases out of ER particulates, pellets or beads more quickly than does an antihistamine (e.g., chlorpheniramine) or antitussive (e.g., hydrocodone). For example, such formulations may comprise the antihistamine, antitussive and pseudoephedrine in ER particulates, pellets or beads, while the IR liquid/vehicle portion comprises the antihistamine and antitussive, but not pseudoephedrine. Certain such formulations still exhibit IR and ER release of all three drugs in a manner that is bioequivalent to the release of the drugs upon administration of corresponding RLDs dosed two or more times as directed on FDA approved labeling, for example over 12 hours.

In certain embodiments, inactive ingredients serving as a carrier for the APIs in formulations of the present invention include: ammonio methacrylate copolymer, lactose monohydrate, methylparaben, microcrystalline cellulose, propylparaben, purified water, sodium alginate, sucrose, talc, titanium dioxide, triethylcitrate. Inert components, such as these, may be used to prepare two distinct phases in formulations of the present invention: a dispersion medium, containing immediate release versions of the drugs, dissolved in syrup; and a dispersed phase comprising coated particulates, pellets or beads containing the extended release portions of the drugs.

Bioequivalence (BE) is a pharmacokinetics term used to describe the in vivo biological equivalence of two preparations of a drug. A bioequivalence requirement refers to a requirement imposed by the FDA for in vitro and/or in vivo testing of specified drug products that must be satisfied as a condition of marketing, under 21 C.F.R. §320.1(f). The U.S. Food and Drug Administration (FDA) has defined bioequivalence as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.” 21 C.F.R. §320.1(e); see also “Bioavailability and Bioequivalence Studies for Orally Administered Drug Products—General Considerations” published by FDA Jul. 2, 2002, at www.fda.gov/OHRMS/DOCKETS/98fr/02d-0258-gd10001.pdf; formally adopted Mar. 19, 2003 (68 Fed. Reg. 13316 (Mar. 19, 2003)).

BE can be measured by comparing the appropriate pharmacokinetic parameters between the two drugs and determining if they fall within an acceptable limit. “Bioequivalent” serum levels of an active ingredient means that the average log transformed values of AUC_{infinity} measured during a single dose study, and Cₘₐₓ Cₘᵢₙ, and AUC_{infinity} measured during a steady state study for the active ingredient, as measured in the serum of a patient after administration of a first drug product comprising that active ingredient, is within 80 to 125 percent of the Cₘₐₓ, Cₘᵢₙ and AUC_{infinity} for the active ingredient, as measured in the serum after administration of a second drug product comprising the active ingredient, within a 90% confidence interval.

The FDA recommends a logarithmic transformation of the pharmacokinetic parameters before statistical analysis is done to determine BE. The traditional FDA-recommended BE limits are that the log transformed PK parameters must be within 80 to 125 percent of each other, within a 90% confidence limit. In one embodiment herein, the novel formulation is a 12 hour controlled release drug. This product may be compared to two or three doses of the RLDs dosed two to three times over a 12 hour period, once at t=0 and once at t=6 hours, or once at t=0, once at t=4 hours and once at t=8 hours.

A single dose study is a study in which an ER product of interest is given to patients only once, and its corresponding IR reference listed drugs (RLDs) are dosed for an equivalent 12 hour dose as directed by the FDA approved label on the IR RLDs. A steady state study is a study in which the ER product of interest and IR RLDs are given repeatedly over time during the study until a steady-state blood serum level of the APIs are achieved. The phrase “Cₘₐₓ” refers to the highest serum concentration (e.g., ng/ml) observed in a patient after administration after steady state has been reached. The phrase “Cₘᵢₙ” refers to the lowest serum concentration (e.g., ng/ml) observed in a patient after steady state for the drug has been reached. The phrase “AUC_{infinity}” or “AUC” refers to the area (e.g., ng/ml×hr) under a curve that plots the concentration of an active ingredient in serum over time, from time 0 to infinity, after administration of one or more doses of a drug product over a time period (e.g., 8, 12, 24, 48 hrs, etc.). “Cₘᵢₙ,” “Cₘₐₓ” and “AUC_{infinity}” for an active ingredient may be measured by well known methods.

“Reference listed drug” or “RLD” refers to a listed drug identified by FDA as a drug product upon which an applicant may rely in seeking approval of an abbreviated new drug application (ANDA). Thus, single drug containing RLDs are defined by the FDA. For generic drugs or drugs filed under a 505(b)(2) application, if there is more than one supplier for an API, the FDA selects which supplier will provide the product acting as the RLD. For the purpose of the present invention, RLDs include separate IR drug products containing a single active ingredient. RLDs may be dosed in combination, for example, by administering sequentially per their dosing instructions, for purposes of comparing to formulations of the present invention. Likewise, for the purposes of the present invention, the term “reference listed drug” or “RLD” also refers to a cocktail containing two or more single drug RLDs. For example, an RLD may be a cocktail containing an antihistamine RLD, an antitussive RLD, and a decongestant RLD. For chlorpheniramine, the FDA-recognized RLD is Chlor-Trimeton Syrup. For PSE, the FDA-recognized RLD is Sudafed Syrup. For Hydrocodone, the FDA-recognized RLD is Hycodan Syrup. When comparing serum levels obtained upon administration of a formulation of the present invention and evaluating bioequivalence, one may administer to a human a cocktail containing all three single drug RLDs in a way that complies with FDA-approved labeling for all of the RLDs.

In January 2001, FDA released guidelines describing bioequivalence studies generally, how to set up such studies, how to analyze data, etc., in a document entitled “Guidance for Industry: Statistical Approaches to Establishing Bioequivalence” (“Statistical Approaches”). In this document, the FDA defined the standards that it intended to use to determine if a product has achieved the statutory definition of BE. When referring herein to a product of interest being BE to its reference drug, the definition of “average BE” as described in these documents applies.

As described on page 2 of “Statistical Approaches,” the FDA recommends that “a standard in vivo BE study design be based on the administration of either single or multiple doses of the T (test) and R (reference) products to healthy subjects on separate occasions, with random assignment to the two possible sequences of drug product administration . . . [and that] statistical analysis for pharmacokinetic measures, such as area under the curve (AUC) and peak concentration (Cₘₐₓ), be based on the two one-sided tests procedure to determine whether the average values for the pharmacokinetic measures determined after administration of the T and R products were comparable. This approach is termed average bioequivalence and involves the calculation of a 90% confidence interval for the ratio of the averages (population geometric means) of the measures for the T and R products. To establish BE, the calculated confidence interval should fall within a BE limit, usually 80-125% for the ratio of the product averages.” “Statistical Approaches,” Section II.B, page 2 (emphasis in original).

The statistical analysis of BE data is based on a statistical model for the log transform of the bioavailability data (e.g., AUC, Cₘₐₓ, Cₘᵢₙ). The “Statistical Approaches” guidance suggests that BE measures be log transformed (either natural log or base 10). For data analysis, “Statistical Approaches” recommends using parametric (normal theory) methods for the analysis of log-transformed BE measures. “For average BE using the criterion stated in equations 2 or 3 (section [IV.A]), the general approach is to construct a 90% confidence interval for the quantity and to reach a conclusion of average BE if this confidence interval is contained in the interval [−θ_{A}, θ_{A}] . . . . The 90% confidence interval for the difference in the means of the log-transformed data should be calculated using methods appropriate to the experimental design.” “Statistical Approaches,” Section VI.B, page 10.

Embodiments of the present invention integrate the benefits of three generally recognized as safe and effective (GRASE) APIs, i.e., an antihistamine, an antitussive, and a decongestant, into one ER medicine. Currently, when used together such drugs are dosed 4-6 times daily in their IR forms because they are not available in a single OTC or prescription-controlled extended release triple-acting combination product. Thus, IR/ER formulations comprising an antihistamine, an antitussive, and a decongestant, where the formulation exhibits ER release of all three drugs upon administration of the single product, provides advantages over currently available products that either do not contain all three active ingredients and/or are merely IR products. For example, upon using the formulations of the current invention, patients will require lower volumes of medicine, e.g., 5 ml as compared to 50 to 55 ml per day of IR products, to achieve relieve from symptoms of a cold, flu or allergy. Formulations of the present invention may also be sold in convenient unit-of-dose 4 ounce containers. Patients will also need to take medicine less often, and run a smaller risk of missing necessary doses to maintain relief over the course of a day. Given that patient compliance is an ever-present and well-recognized problem, a formulation that provides bioequivalent doses of all three drugs in a single dose (e.g. 12 hour dose) offers significant advantages over presently available formulations.

In one embodiment, the present invention provides a formulation comprising a novel mixture of IR and ER forms of chlorpheniramine, pseudoephedrine and hydrocodone within a single product, where the single product is administered to a patient less often, while achieving bioequivalence, in patients administered immediate release product(s) containing these drugs.

The present invention also relates to drug combination formulations and methods of manufacturing such formulations, such as stable oral extended release drugs in a liquid suspension or solid capsules or tablets, that comprise particulates, pellets, or beads having two or more active ingredients contained within each single particulate, pellet, or bead. This approach has multiple advantages not only over IR formulations, but also over ER formulations.

In other embodiments, the invention provides a novel oral liquid suspension formulation comprising an extended-release component comprising pellets, beads or particles containing one or more drugs, where the pellets, beads or particles are suspended in a syrup. The syrup may also contain one or more drugs. The present specification provides an example of such an oral liquid suspension. The oral liquid suspension formulation achieves superior properties over the prior art. For example, because the ER component of the example formulation comprises beads comprising three drugs associates with an ion-exchange matrix, and those beads are suspended in a syrup, the formulation provides greatly increased product stability over other liquid formulations. Typically, drug degradation occurs in an aqueous environment. While degradation can be minimized by the use of solid dosage forms, this prevents the ease of dosing and dosage flexibility found in liquid formulations. In at least one embodiment, the present inventive dosage form minimizes exposure to water by a two step approach: (1) the beads exhibit ER properties, rather than being readily soluble in a liquid phase used to suspend the beads; and (2) the liquid phase is a syrup, where the presence of sugars lower the water activity of the liquid phase. The syrup, by decreasing water activity and increasing osmotic pressure, also serves to: (a) minimize leaching of drugs from the ER beads into the syrup; and (b) prevents degradation of the beads. Conversely, when consumed by a patient, the beads are then able to release the drugs, for example by the beads swelling and degrading in the intestines, allowing for drug release from the ER.

The concentration of ion-exchange matrix resin drug complex in the liquid form controlled release drug composition of the invention consisting of chlorpheniramine, hydrocodone and pseudoephedrine as active ingredients can vary over a wide range depending, e.g., on the particular drug, the content of drug in the of ion-exchange matrix resin drug complex; the condition or symptom to be treated; and the age of the patient. In one embodiment of such composition, the concentration of ion-exchange matrix resin drug complex in the liquid form controlled release drug composition ranges from about 5% to about 90% by weight based on the total weight of the liquid form controlled release drug composition; in the another embodiment of such composition, the weight of ion-exchange matrix resin drug complex ranges from about 10% to about 50% based on the based on the total weight of the liquid form controlled release drug composition; and in the another embodiment of such composition, the weight of ion-exchange matrix resin drug complex ranges from about 20% to about 40% based on the based on the total weight of the liquid form controlled release drug composition.

In an embodiment of the drug composition of the invention consisting of chlorpheniramine, hydrocodone and pseudoephedrine as active ingredients, the method of preparing the dispersed phase comprises mixing of a drug in a powder form and an ion-exchange matrix in a powder form. In such embodiment, salt forms of the drug and the ion-exchange matrix may be used. The powder blending method of preparing the dispersed phase of the present invention is cost and time-effective as compared to conventional prior art methods.

In one embodiment of the drug composition of the invention consisting of chlorpheniramine, hydrocodone and pseudoephedrine as active ingredients, the ion exchange matrix is sodium alginate. In one but non-limiting embodiment, powders of chlorpheniramine maleate, pseudoephedrine hydrochloride and hydrocodone bitartrate alone or in combination may be mixed with sodium alginate powders. In another embodiment, lactose, microcrystalline cellulose and/or other excipients may be added to the mixture of the drugs and ion-exchange powders.

In another embodiment of the drug composition of the invention consisting of chlorpheniramine, hydrocodone and pseudoephedrine as active ingredients, subsequent to the drug and ion-exchange powder blending, wet massing is continued with the addition of water, followed by extrusion and spheronization. The resulting core beads or pellets containing drug and ion-exchange matrix may be dried in a fluid bed dryer. In one such embodiment, each bead or pellet comprises several active ingredients associated with the same ion-exchange matrix. For example, each bead or pellet may comprise chlorpheniramine maleate, pseudoephedrine hydrochloride and hydrocodone bitartrate associated with sodium alginate powders.

In certain embodiments of the drug composition of the invention consisting of chlorpheniramine, hydrocodone and pseudoephedrine as active ingredients, the resulting beads are coated with EUDRAGIT® in the presence of triethyl citrate and talc in a fluid bed processor. Then, the coated beads are blended with talc and cured in an oven. In one such embodiment, coated beads are cured for 2 h, 4 h, 8 h, 16 h, 24 h, or 48 h. In certain embodiments, coated beads are cured from about 16 hours to about 24 hours. The curing time of the coated beads may have an effect on a rate of release of the drug from the coated beads.

The coated beads consisting of chlorpheniramine, hydrocodone and pseudoephedrine as active ingredients are suspended in a dispersion medium that comprises salt forms of drugs, water and sucrose. In one but non-limiting embodiment of such composition of the invention, the dispersion medium comprises chlorpheniramine maleate and hydrocodone bitartrate. In another embodiment of the composition of the invention, the dispersion medium also comprises pseudoephedrine hydrochloride. The dispersion medium can also further comprise preservatives, taste masking agents and other non-active additives. In such embodiments, the resulting liquid sustained release product is capable of maintaining physical and chemical stability in a bottle, and capable of achieving controlled release of drug product when administered to a patient.

In one embodiment, the present invention relates to a method for preparing a liquid form controlled release drug composition consisting of chlorpheniramine, hydrocodone and pseudoephedrine as active ingredients, comprising:
(a) preparing the dispersed phase, which comprises preparing particulates, pellets or beads, wherein active ingredients consisting of chlorpheniramine, hydrocodone and pseudoephedrine associate in a single particulate, pellet or bead;
(b) preparing the dispersion medium, wherein the dispersion medium comprises active ingredients consisting of chlorpheniramine, hydrocodone and optionally pseudoephedrine;
(c) coating the particulates, pellets or beads with a membrane coating; and
(d) dispersing the beads into a dispersion medium.

In one embodiment of the above-described method, the step of preparing the dispersed phase further comprises associating chlorpheniramine, hydrocodone and pseudoephedrine with a pharmaceutically acceptable ion-exchange matrix. In another embodiment, the step of preparing the dispersed phase further comprises preparing particulates, pellets or beads consisting of chlorpheniramine, hydrocodone and pseudoephedrine and a pharmaceutically acceptable ion-exchange matrix, wherein chlorpheniramine, hydrocodone and pseudoephedrine bind to the ion-exchange matrix in a single particulate, pellet or bead. In another embodiment, the step of preparing the dispersed phase further comprises blending of chlorpheniramine, hydrocodone and pseudoephedrine and ion exchange matrix powders. In yet another embodiment, the step of preparing the dispersed phase further comprises wet granulation, extrusion and spheronization of chlorpheniramine, hydrocodone and pseudoephedrine and ion exchange matrix powders. In one embodiment, salt forms of the chlorpheniramine, hydrocodone and pseudoephedrine and the ion exchange matrix are used.

One advantage is that the present formulation achieves bioequivalence for two or more drugs at the same time. Comparative products may not have the same release profile and require different doses or frequency of dosing for each drug. In addition, if different ER technologies are used (e.g. different resins), then the release profile of each drug may be affected by differences in a patient's diet or physiology, such that a given patient may receive too much of one drug, but too little of another. By placing three drugs, for example, in a single bead with a single release technology, the drug release profile will be more consistent across the patient population.

If each of the three drugs are placed in separate beads, which are then mixed, it is possible that the mixture will not be perfectly homogenous, especially in small amounts. Such non-homogeneity will result in incorrect relative doses for the drugs: too high for some, too low for others. Non-homogeneity can occur either due to random fluctuations, but may also be due to different physical properties of the three classes of beads in a three bead class mixture. For example, pseudoephedrine constitutes a larger proportion (by weight) of the drugs. If all three drugs are packaged in individual beads of equivalent size and amount, the greater amount of pseudoephedrine relative to excipient will result in a different density of the pseudoephedrine bead over a hydrocodone or chlorpheniramine bead. Such a density difference would result in a loss of homogeneity. Even if beads are calibrated to be of equivalent density at a given temperature and pressure, this may not hold true under different conditions.

Density difference can also be used to deliberately separate beads according to drug class, and thereby concentrate (say) pseudoephedrine, for diversion into illegal drug use. On the other hand, if all drugs are in a single type of bead, it is not possible to partially isolate one drug from others on the basis of differences in the physical properties of the beads. Moreover, the production of such products will significantly deter or prevent drug abuse or diversion of any one active ingredient present in the particulate, pellet, or bead. By combining or infusing two or more drugs within a single particulate, pellet, or bead, individuals cannot easily extract or separate out individual active ingredients from the products for abuse. In addition, by combining or infusing two or more active ingredients into single particulate, pellet, or bead, one reduces the surface area of particulates, pellets, or beads present in an overall drug combination product, thereby providing increased stability of the product and active ingredients.

In one embodiment, a drug combination product will have a decreased potential for separation or isolation of a single active ingredient by comprising particulates, pellets, or beads having two or more active ingredients per particulate, pellet, or bead. Likewise, the present invention provides methods for manufacturing cold and allergy combination drug formulations that have less abuse potential with regard to any single ingredient included in the formulation. For example, each bead within a product may comprise a pharmaceutically acceptable ion-exchange matrix and two or more pharmaceutically acceptable active ingredient drugs associated with the ion-exchange matrix, as such as those prepared using technology described in published patent applications owned by UPM Pharmaceuticals (see U.S. Ser. No. 10/724,276, U.S. Ser. No. 11/150,572 and U.S. Ser. No. 11/198,937, hereby incorporated by reference), relating to the production and use of a certain type of ER beads in suspension. From such a product, one cannot easily extract, separate or isolate any single active ingredient drug from the drug combination product in a makeshift laboratory.

Thus, formulations of the current invention differ from others currently available in the cough/cold market. For example, Tussionex® Extended-Release Suspension is a cough-suppressant/antihistamine combination, comprising hydrocodone and chlorpheniramine, used to relieve coughs and the upper respiratory symptoms of colds and allergies. This liquid suspension product contains drug-ion exchange resin beads, where any individual beads in the suspension is impregnated with either hydrocodone or chlorpheniramine, but not both drugs in any single bead.

Formulations of the present invention will also differ from those currently available in that one will not be able to readily rely on common household products and easily assessable equipment, or “recipes” for making or isolating a drug of abuse via the Internet, with formulations of the present invention. Rather, separation of a potential drug of abuse of interest from formulations of the present invention will require high quality state of the art equipment and scientific training and technology, e.g., complex chromatography, normally only available in academic or industrial laboratories.

In another embodiment of the present invention, a drug combination product has a decreased potential for abuse and/or diversion by comprising ion-exchange matrix drug particulates, pellets, or beads. Each bead may comprise a pharmaceutically acceptable ion-exchange matrix and two or more pharmaceutically acceptable active ingredient drugs associated with the ion-exchange matrix.

The invention also includes methods for preventing or reducing abuse of at least one active ingredient, comprising preparing a drug combination product, wherein the product comprises particulates, pellets, or beads, wherein each particulate, pellet, or bead comprises a pharmaceutically acceptable ion-exchange matrix and two or more pharmaceutically acceptable active ingredient drugs associated with the ion-exchange matrix.

In another embodiment, methods for preventing or reducing the ability to extract, isolate or separate out a single active ingredient comprise preparing a drug combination product, wherein that product comprises particulates, pellets, or beads, wherein each particulate, pellet, or bead comprises a pharmaceutically acceptable ion-exchange matrix and two or more pharmaceutically acceptable active ingredient drugs associated with the ion-exchange matrix. The fact that the matrix particulate, pellet, or bead comprises two or more active ingredients makes extraction or separation of any single drug exceedingly difficult for an untrained individual in a makeshift laboratory lacking industrial grade or other high quality equipment, such as chromatography equipment.

In another embodiment, drug combination products of the present invention allow for appropriate and precise dosing by a patient of, for example, three different active ingredients. When patients self-medicate with multiple compositions (e.g., three different products, where each contains a single IR active ingredient), appropriate and precise dosing is often difficult, especially with regard to avoiding over- or under-dosing of one or more drugs, and/or maximizing therapeutic benefit of all drugs while minimizing side effects. Combination products of the present invention avoid such difficulties because all relevant drugs are supplied and administered in single dose forms, such as in a 12-hour ER form.

In one embodiment, the drug combination product is a liquid suspension composition comprising a dispersed phase comprising coated ion-exchange matrix drug particulates, pellets, or beads containing extended released drugs, optionally comprising a dispersion medium containing immediate release drugs, dissolved in syrup.

In one embodiment, the dosage of the controlled release drug composition used is the dosage sufficient to achieve a therapeutic effect in a patient, for example, in a human patient, wherein the term “therapeutic effect” means any effect against a cold, flu or an allergy, including but not limited to symptomatic relief, such as reducing severity and/or frequency of coughing, symptoms of coughing, nasal discharge, congestion or sneezing, and/or other biological effects resulting in an improvement in subjective well-being.

In one specific embodiment, the present invention relates to a non-liquid oral extended release drug composition comprising a first portion and a second portion, wherein

the first portion comprises an antihistamine, an antitussive, and optionally a decongestant as active ingredients in an immediate release form,

the second portion comprises a particulate, pellet, or bead that comprises the antihistamine, the antitussive, and the decongestant as three active ingredients in an extended release form,

administration of a single dose of the non-liquid oral drug composition to a patient provides serum levels of the three active ingredients over a time period of at least 8 hours that are bioequivalent to serum levels achieved upon administration of an appropriate number of doses, over the same time period, of FDA-approved immediate release reference listed drug (IR RLD) compositions comprised of the active ingredients, and

the appropriate number of doses corresponds to a number of doses recommended in one or more FDA-approved labels for administration of the IR RLD compositions over the same time period.

In one embodiment of the above-described composition, the antitussive is a narcotic antitussive. In another embodiment of the above-described composition, the first portion does not comprise the decongestant, and comprises only an antihistamine and an antitussive. In a specific embodiment the antihistamine is chlorpheniramine. In another specific embodiment, the antitussive is hydrocodone. In yet another specific embodiment, the decongestant is pseudoephedrine. In one embodiment of the above-described composition, the particulate, pellet or bead further comprises a coating.

In one embodiment of the above-described composition, the antihistamine, the antitussive and the decongestant associate in a single particulate, pellet or bead. In such embodiment, the particulate, pellet or bead further comprises a pharmaceutically acceptable ion-exchange matrix, wherein the antihistamine, the antitussive and the decongestant associate with the ion-exchange matrix.

Some embodiments of the above-described composition further comprise an excipient selected from the group consisting of sweetening agents, flavoring agents, coloring agents, and any combination thereof. Other embodiments further comprise an additive selected from the group consisting of stabilizing agents, dispersion agents, and any combination thereof.

In a certain embodiment, the present invention envisages a non-liquid oral extended release drug composition comprising a first portion and a second portion, wherein

the first portion comprises an antihistamine, an antitussive, and optionally a decongestant, as active ingredients in an immediate release form,

the second portion is a particulate, pellet or bead that comprises the antihistamine, the antitussive, and the decongestant as active ingredients in an extended release form,

administration of a sufficient number of doses of the non-liquid drug composition to a patient to achieve steady-state serum levels of the three active ingredients over a time period of greater than 24 hours yields serum levels of the active ingredients that are bioequivalent to serum levels achieved upon administration of an appropriate number of doses, over the same time period, of one or more FDA-approved immediate release drug compositions comprised of the active ingredients, and

the appropriate number of doses corresponds to a number of doses recommended in one or more FDA-approved labels for the administration of the one or more FDA-approved immediate release drug compositions over the same time period.

In another embodiment, the present invention relates to a method for treating cough, cold, flu or allergy symptoms in a human subject, comprising the step of administering the non-liquid oral extended release drug composition described herein to the subject. In one such embodiment, the pharmaceutical composition is administered as a dual release formulation allowing a one-a-day or twice-a-day dosing in humans.

In yet another embodiment, the present invention relates to a method of treating coughing, symptoms of coughing, nasal discharge, congestion or sneezing associated with a cold, flu or an allergy for a time period of at least 8 hours, comprising administering to a human subject in need of such a treatment a single dose of the non-liquid drug composition described herein effective to treat coughing, symptoms of coughing, nasal discharge, congestion or sneezing associated with a cold or an allergy, for a time period of at least 8 hours.

In yet another embodiment, the present invention relates to a method for making the non-liquid oral extended release drug composition described herein, comprising

preparing the immediate release portion, wherein the immediate release portion comprises an antihistamine, an antitussive, and optionally a decongestant as active ingredients;

preparing the extended release portion, which comprises preparing particulates, pellets or beads comprising an antihistamine, an antitussive, and a decongestant as active ingredients;

coating the particulates, pellets or beads with a membrane coating; and

combining the extended release portion with the immediate release portion.

In certain embodiments, the above-described method further comprises preparing particulates, pellets or beads, wherein two or more active ingredients associate in a single particulate, pellet or bead. In one specific embodiment, such method further comprises the step of associating the two or more active ingredients with a pharmaceutically acceptable ion-exchange matrix. In a specific embodiment the antihistamine is chlorpheniramine. In another specific embodiment, the antitussive is hydrocodone. In yet another specific embodiment, the decongestant is pseudoephedrine.

In another embodiment, the present invention relates to a method for achieving in a mammal serum levels of an antihistamine, an antitussive and a decongestant over a time period of at least 8 hours that are bioequivalent to serum levels achieved upon administration of an appropriate number of doses over the same time period of FDA-approved immediate release reference listed drug (IR RLD) compositions to the same mammal, wherein the method comprises:

administering to the mammal a non-liquid oral extended release drug composition comprising a first portion and a second portion, wherein the first portion comprises the antihistamine, the antitussive and optionally the decongestant as active ingredients in an immediate release form, and wherein the second portion comprises a particulate, pellet, or bead that comprises the antihistamine, antitussive and the decongestant as active ingredients in an extended release form, and

achieving serum levels of the three active ingredients over a time period of at least 8 hours that are bioequivalent to serum levels achieved upon administration of an appropriate number of doses over the same time period of FDA-approved IR RLD compositions comprising the active ingredients, wherein the appropriate number of doses corresponds to a number of doses recommended in one or more FDA-approved labels for the administration of the IR RLD compositions over the same time period.

In yet another embodiment, the present invention relates to a method for achieving in a mammal steady-state serum levels of an antihistamine, an antitussive and a decongestant upon administration of a non-liquid oral extended release (ER) drug composition, wherein the serum levels are bioequivalent to serum levels achieved upon administration of one or more immediate release (IR) compositions comprising active ingredients and inactive ingredients, wherein said active ingredients consist of chlorpheniramine, hydrocodone and pseudoephedrine to the same mammal, wherein the method comprises:

administering to the mammal a non-liquid oral ER drug composition comprising a first portion and a second portion, wherein the first portion comprises the antihistamine, the antitussive and the decongestant as active ingredients in an immediate release form, and wherein the second portion comprises a particulate, pellet or bead that comprises the antihistamine, the antitussive and the decongestant as active ingredients in an extended release form, and

wherein administration of a sufficient number of doses of the non-liquid oral ER drug composition to the mammal to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release (IR) drug compositions comprising the active ingredients,

wherein the appropriate number of doses of the one or more FDA-approved IR drug compositions corresponds to a number of doses recommended in one or more FDA-approved labels for the administration of the one or more FDA-approved IR drug compositions over the same time period, and

wherein the appropriate number of doses of the one or more FDA-approved IR drug compositions is greater than the sufficient number of doses of the oral ER drug composition.

In one embodiment, the present invention relates to a non-liquid oral extended release drug composition comprising a first portion and a second portion, wherein

the first portion comprises chlorpheniramine, hydrocodone, and optionally pseudoephedrine as active ingredients in an immediate release form,

the second portion comprises a particulate, pellet, or bead that comprises chlorpheniramine, hydrocodone and pseudoephedrine as three active ingredients in an extended release form,

administration of a single dose of the non-liquid oral drug composition to a patient provides serum levels of the three active ingredients over a time period of at least 8 hours that are bioequivalent to serum levels achieved upon administration of an appropriate number of doses over the same time period of FDA-approved immediate release reference listed drug (IR RLD) compositions comprising the active ingredients, and

the appropriate number of doses corresponds to a number of doses recommended in one or more FDA-approved labels for the administration of the IR RLD compositions over the same time period.

In some embodiments of the above-described drug composition, the time period of at least 8 hours is 12 hours. In other embodiments, the time period of at least 8 hours is 24 hours.

In some embodiments of the above-described drug composition, the first portion does not comprises pseudoephedrine.

In one embodiment, the drug composition is in an oral solid form. In yet another embodiment, the drug composition is in an oral capsule form.

In some embodiments of the above-described drug composition, the particulate, pellet or bead further comprises a coating. In some such embodiments, the particulate, pellet or bead further comprises a pharmaceutically acceptable ion-exchange matrix, wherein the chlorpheniramine, hydrocodone and pseudoephedrine associate with the ion-exchange matrix.

In certain embodiments, the above-described drug composition further comprises an excipient selected from the group consisting of sweetening agents, flavoring agents, coloring agents, and any combination thereof. In certain embodiments, the drug composition further comprises an additive selected from the group consisting of stabilizing agents, dispersion agents, and any combination thereof.

In some embodiments of the above-described drug composition, the time period of at least 8 hours is 12 hours, and the drug composition comprises active ingredients that consist of 8 to 12 mg chlorpheniramine maleate, 10 to 15 mg hydrocodone bitartrate and at least 120 mg pseudoephedrine per 5 ml single dose. In other embodiments, the time period of at least 8 hours is 24 hours, and the drug composition comprises active ingredients that consists of 16 to 24 mg chlorpheniramine maleate, 20 to 30 mg hydrocodone bitartrate and at least 240 mg pseudoephedrine hydrochloride per 5 ml single dose.

In another embodiment, the present invention envisages a method for treating coughing, symptoms of coughing, nasal discharge, congestion or sneezing associated with a cold, flu or an allergy for a time period of at least 8 hours, comprising administering to a human subject in need of such a treatment a single dose of the drug composition that consists of chlorpheniramine, hydrocodone and pseudoephedrine as active ingredients effective to treat coughing, symptoms of coughing, nasal discharge, congestion or sneezing associated with a cold or an allergy, for the time period of at least 8 hours.

In certain embodiments of the above-described method, the time period of at least 8 hours is 12 hours. In another embodiment of such method, the time period of at least 8 hours is 24 hours.

In a certain embodiment, the present invention encompasses a non-liquid oral pharmaceutical formulation comprising chlorpheniramine, hydrocodone and pseudoephedrine as active ingredients, wherein the formulation exhibits immediate release (IR) and extended release (ER) of the active ingredients, wherein

the formulation comprises an immediate release portion and an extended release portion, and

administration of a single dose of the non-liquid oral formulation to a patient provides serum levels of chlorpheniramine, hydrocodone and pseudoephedrine over a time period of at least 8 hours that are bioequivalent to serum levels achieved upon administration of two or more doses, over the same time period, of one or more IR compositions comprising chlorpheniramine, hydrocodone and/or pseudoephedrine.

In one embodiment of the above-described formulation, the time period of at least 8 hours is 12 hours. In another embodiment, the time period of at least 8 hours is 24 hours.

In certain embodiments, the present invention relates to a method of making the non-liquid oral pharmaceutical formulation described above, comprising preparing the immediate release portion, wherein the immediate release portion comprises chlorpheniramine and hydrocodone, but not pseudoephedrine.

In other embodiments, the present invention contemplates a method of making the non-liquid oral pharmaceutical formulation described above, comprising preparing the extended release portion, which comprises preparing particulates, pellets or beads, wherein each individual particulate, pellet or bead comprises chlorpheniramine, hydrocodone and pseudoephedrine,

wherein the method further comprises combining the extended release portion with the immediate release portion.

In some embodiments, the above-described method, further comprises coating the particulates, pellets or beads with a membrane coating prior to combining the extended release portion with the immediate release portion.

In certain embodiments, the present invention relates to a non-liquid oral extended release drug composition comprising a first portion and a second portion, wherein

the first portion comprises chlorpheniramine, hydrocodone, and optionally pseudoephedrine as active ingredients in an immediate release form,

the second portion is a particulate, pellet or bead that comprises chlorpheniramine, hydrocodone and pseudoephedrine as active ingredients in an extended release form,

administration of a sufficient number of doses of the non-liquid drug composition to a patient to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release drug compositions comprising the active ingredients, and

the appropriate number of doses corresponds to a number of doses recommended in one or more FDA-approved labels for the administration of the one or more FDA-approved immediate release drug compositions over the same time period.

In another embodiment, the present invention relates to a non-liquid oral extended release drug composition comprising a first portion and a second portion, wherein

the first portion comprises chlorpheniramine, hydrocodone, and optionally pseudoephedrine as active ingredients in an immediate release form,

the second portion comprises a particulate, pellet, or bead that comprises chlorpheniramine, hydrocodone and pseudoephedrine as active ingredients in an extended release form,

administration of a single dose of the non-liquid drug composition to a patient provides serum levels of the three active ingredients over a time period of at least 8 hours that are bioequivalent to serum levels achieved upon administration of an appropriate number of doses over the same time period of an FDA-approved immediate release reference listed drug (IR RLD) composition comprising all three active ingredients, and

the appropriate number of doses corresponds to a number of doses recommended in an FDA-approved label for the administration of the IR RLD composition over the same time period.

In yet another embodiment, the present invention relates to a non-liquid oral pharmaceutical composition comprising: (1) an immediate release (IR) portion comprising chlorpheniramine and hydrocodone as active ingredients, and (2) an extended release (ER) portion comprising chlorpheniramine, hydrocodone and pseudoephedrine as active ingredients, wherein

the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

administration of a single dose of the non-liquid oral composition provides an AUC_{infinity} for hydrocodone in a human subject that is equivalent to an AUC_{infinity} obtained upon administration of two or more doses of an immediate release reference listed drug (IR RLD) having one half or less of the amount of hydrocodone present in the oral composition, and

administration of a single dose of the non-liquid oral composition provides an AUC_{infinity} for pseudoephedrine in a human subject that is equivalent to an AUC_{infinity} obtained upon administration of two or more doses of an immediate release reference listed drug (IR RLD) having one half or less of the amount of pseudoephedrine present in the oral composition.

In some embodiments of the non-liquid oral composition described above, administration of a single dose of the oral composition provides an AUC_{infinity} for chlorpheniramine in a human subject that is equivalent to an AUC_{infinity} obtained upon administration of two or more doses of an immediate release reference listed drug (IR RLD) having one half or less of the amount of chlorpheniramine present in the oral composition.

In certain other embodiments, the present invention relates to a non-liquid oral pharmaceutical composition comprising: (1) an immediate release (IR) portion comprising chlorpheniramine and hydrocodone as active ingredients, and (2) an extended release (ER) portion comprising chlorpheniramine, hydrocodone and pseudoephedrine as active ingredients, wherein

the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

the non-liquid oral composition demonstrates an AUC_{infinity} for hydrocodone in a human subject that is equivalent to an AUC_{infinity} obtained upon administration of two doses of an immediate release reference listed drug (IR RLD) having one half the amount of hydrocodone as compared to the oral composition, wherein the oral composition is dosed once, and the IR RLD is dosed twice at zero and six hours, over a 12 hour period, and

the non-liquid oral composition demonstrates an AUC_{infinity} for pseudoephedrine in a human subject equivalent to an AUC_{infinity} obtained upon administration of two doses of an IR RLD having one half the amount of pseudoephedrine as compared to the oral composition, wherein the oral composition is dosed once, and the IR RLD is dosed twice at zero and six hours, over a 12 hour period.

In some specific embodiments, the above-described non-liquid oral composition demonstrates an AUC_{infinity} for chlorpheniramine in a human subject equivalent to an AUC_{infinity} obtained upon administration of two doses of an IR RLD having one half the amount of chlorpheniramine as compared to the oral composition, wherein the oral composition is dosed once, and the IR RLD is dosed twice at zero and six hours, over a 12 hour period.

In one embodiment the present invention envisages a method for treating cough, cold, flu or allergy symptoms in a human subject, comprising the step of administrating one of the non-liquid oral extended release drug compositions described herein to the subject.

In certain embodiments of the above-described method, the pharmaceutical composition is administrated as a dual release formulation allowing a one-a-day or twice-a-day dosing in humans.

In some embodiments, the present invention relates to a non-liquid oral extended-release drug composition comprising an antihistamine, an antitussive and a decongestant as active ingredients, wherein the composition provides sufficient AUC_{infinity} of all three active ingredients to achieve a therapeutic effect for a time period of at least 8 hours after a single dose in a human subject, according to serum analysis.

In one specific embodiment, the present invention relates to a non-liquid oral extended-release drug composition comprising chlorpheniramine, hydrocodone and pseudoephedrine as active ingredients, wherein the composition provides sufficient AUC_{infinity} of all three active ingredients to achieve a therapeutic effect for a time period of at least 8 hours after a single dose in a human subject, according to serum analysis.

In the context of the embodiments described above, the term “therapeutic effect” means any effect against a cold, flu or an allergy, including but not limited to symptomatic relief, such as reducing severity and/or frequency of coughing, symptoms of coughing, nasal discharge, congestion or sneezing, and/or other biological effects resulting in an improvement in subjective well-being.

In one embodiment of the above-described non-liquid drug composition, the time period of at least 8 hours is 12 hours. In yet another embodiment of the above-described non-liquid drug composition, the time period of at least 8 hours is 24 hours.

In one embodiment the present invention encompasses a method for preventing or reducing an ability to extract, isolate or separate out pseudoephedrine or ephedrine present in a non-liquid oral extended-release drug composition, comprising:

preparing the non-liquid oral extended release drug composition so that it comprises a first portion and a second portion, wherein

the first portion comprises an antihistamine, an antitussive, or both in immediate release form, and optionally comprises pseudoephedrine or ephedrine, and

the second portion comprises particulates, pellets, or beads, wherein each particulate, pellet, or bead comprises pseudoephedrine or ephedrine, and the antihistamine or antitussive or both, as active ingredients in an extended release form, and

preventing or reducing the ability to extract, isolate or separate out pseudoephedrine or ephedrine present in an oral extended-release drug composition.

In one specific embodiment of the above described method, the antitussive is hydrocodone, and wherein the method further comprises preventing or reducing an ability to extract, isolate or separate out hydrocodone present in the non-liquid oral extended-release drug composition.

In another embodiment, the above-described method further comprises a step of manufacturing that makes extraction, isolation or separation of the pseudoephedrine or ephedrine from the non-liquid oral extended-release drug composition more difficult, as compared to an immediate release composition comprising pseudoephedrine or ephedrine.

In certain embodiments the present invention envisages a method of reducing the abuse potential of pseudoephedrine or ephedrine present in a non-liquid oral extended-release drug composition, comprising:

preparing the non-liquid oral extended release drug composition so that it comprises a first portion and a second portion, wherein

the first portion comprises an antihistamine, an antitussive or both in immediate release form, and optionally comprises pseudoephedrine or ephedrine, and

the second portion comprises a particulate, pellet, or bead that comprises the antihistamine, the antitussive, and pseudoephedrine or ephedrine, as active ingredients in an extended release form.

In certain other embodiments the present invention envisages a method for reducing the abuse potential of a narcotic antitussive or pseudoephedrine present in a non-liquid oral extended-release drug composition, comprising preparing the oral extended release drug composition so that it comprises a particulate, pellet, or bead comprising the narcotic antitussive and pseudoephedrine as active ingredients in an extended release form.

In one embodiment the present invention relates to a method for manufacturing a solid oral extended release combination drug formulation for use in the treatment of symptoms of a cold, flu or allergy, wherein the formulation has reduced abuse potential with regard to pseudoephedrine or ephedrine included in the formulation, as compared to an immediate release (IR) formulation comprising pseudoephedrine or ephedrine, wherein the method comprises:

preparing the solid oral extended release drug composition so that it comprises particulates, pellets, or beads, wherein each particulate, pellet, or bead comprises two or more active ingredients in an extended release form, wherein at least one of the active ingredients is pseudoephedrine or ephedrine, and wherein at least one of the active ingredients is not pseudoephedrine or ephedrine.

In another embodiment the present invention relates to a method for preventing or reducing the ability to extract, isolate or separate out pseudoephedrine or ephedrine present in a solid oral extended-release drug composition, comprising:

preparing the solid oral extended release drug composition comprising particulates, pellets, or beads, wherein each particulate, pellet, or bead comprises two or more pharmaceutically acceptable active ingredients in an extended release form, wherein at least one of the active ingredients is pseudoephedrine or ephedrine, and wherein at least one of the active ingredients is not pseudoephedrine or ephedrine.

In yet another embodiment, the present invention contemplates a method for achieving in a mammal serum levels of three active ingredients over a time period of at least 8 hours that are bioequivalent to serum levels achieved upon administration of an appropriate number of doses over the same time period of FDA-approved immediate release reference listed drug (IR RLD) compositions to the same mammal, wherein the method comprises:

(A) administering to the mammal an non-liquid oral extended release drug composition comprising a first portion and a second portion, wherein the first portion comprises an antihistamine, an antitussive, and optionally a decongestant, as active ingredients in an immediate release form, and wherein the second portion comprises a particulate, pellet, or bead that comprises the antihistamine, the antitussive and the decongestant as three active ingredients in an extended release form, and

(B) achieving serum levels of the three active ingredients over a time period of at least 8 hours that are bioequivalent to serum levels achieved upon administration of an appropriate number of doses over the same time period of FDA-approved IR RLD compositions comprising the active ingredients, wherein the appropriate number of doses corresponds to a number of doses recommended in one or more FDA-approved labels for the administration of the IR RLD compositions over the same time period.

In yet another embodiment, the present invention contemplates a method for achieving in a mammal serum levels of chlorpheniramine, hydrocodone and pseudoephedrine over a time period of at least 8 hours that are bioequivalent to serum levels achieved upon administration of two or more doses over the same time period of one or more immediate release (IR) compositions comprising chlorpheniramine, hydrocodone and/or pseudoephedrine to the same mammal, wherein the method comprises:

(A) administering to the mammal a single oral pharmaceutical formulation consisting of chlorpheniramine, hydrocodone and pseudoephedrine as active ingredients, wherein the formulation exhibits IR and extended release (ER) of the active ingredients, wherein the formulation comprises an IR portion and an ER portion, and

(B) achieving serum levels of chlorpheniramine, hydrocodone and pseudoephedrine over a time period of at least 8 hours that are bioequivalent to serum levels achieved upon administration of two or more doses over the same time period of one or more IR compositions comprising chlorpheniramine, hydrocodone and/or pseudoephedrine.

In some embodiments of the above-described method, the time period of at least 8 hours is 12 hours. In other embodiments of the above-described method, the time period of at least 8 hours is 24 hours.

In yet another embodiment, the present invention contemplates a method for achieving in a mammal steady-state serum levels of an antihistamine, an antitussive and a decongestant upon administration of an non-liquid oral extended release (ER) drug composition, wherein the serum levels are bioequivalent to serum levels achieved upon administration of one or more immediate release (IR) compositions comprising the antihistamine, the antitussive and/or the decongestant to the same mammal, wherein the method comprises:

administering to the mammal an non-liquid oral ER drug composition comprising a first portion and a second portion, wherein the first portion comprises an antihistamine, an antitussive, and optionally a decongestant, as active ingredients in an immediate release form, and wherein the second portion is a particulate, pellet or bead that comprises the antihistamine, the antitussive and the decongestant as active ingredients in an extended release form, and

wherein administration of a sufficient number of doses of the oral ER drug composition to the mammal to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release (IR) drug compositions comprising the active ingredients,

wherein the appropriate number of doses of the one or more FDA-approved IR drug compositions corresponds to a number of doses recommended in one or more FDA-approved labels for the administration of the one or more FDA-approved IR drug compositions over the same time period, and

wherein the appropriate number of doses of the one or more FDA-approved IR drug compositions is greater than the sufficient number of doses of the oral ER drug composition.

In certain embodiments the present invention relates to a method for achieving in a mammal serum levels of an antihistamine, an antitussive and a decongestant as active ingredients over a time period of at least 8 hours that are bioequivalent to serum levels achieved upon administration of an appropriate number of doses over the same time period of an FDA-approved immediate release reference listed drug (IR RLD) composition comprising all three active ingredients to the same mammal, wherein the method comprises:

(A) administering to the mammal a single dose of an non-liquid oral extended release (ER) drug composition comprising a first portion and a second portion,

wherein the first portion comprises the antihistamine, the antitussive, and optionally the decongestant, as active ingredients in an IR form,

wherein the second portion is a particulate, pellet, or bead that comprises the antihistamine, the antitussive and the decongestant as active ingredients in an ER form,

(B) achieving serum levels of all three active ingredients over a time period of at least 8 hours that are bioequivalent to serum levels achieved upon administration of an appropriate number of doses over the same time period of an FDA-approved IR RLD composition comprising all three active ingredients, and

wherein the appropriate number of doses corresponds to a number of doses recommended in an FDA-approved label for the administration of the IR RLD composition over the same time period.

In certain other embodiments the present invention relates to a method for achieving AUC_{infinity} values for hydrocodone and pseudoephedrine in a mammalian subject, wherein the method comprises:

(A) administering at the beginning of a time period of at least eight hours a single dose of an extended release (ER) oral pharmaceutical composition comprising: (1) an immediate release (IR) portion consisting of chlorpheniramine and hydrocodone as active ingredients, and (2) an ER portion consisting of chlorpheniramine, hydrocodone and pseudoephedrine as active ingredients,

wherein the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

(B) achieving an AUC_{infinity} for hydrocodone in a mammalian subject that is equivalent to an AUC_{infinity} obtained upon administrating over the same time period two or more doses of an immediate release reference listed drug (IR RLD) having one half or less of the amount of hydrocodone present in the oral composition, and

C) achieving an AUC_{infinity} for pseudoephedrine in a mammalian subject that is equivalent to an AUC_{infinity} obtained upon administrating over the same time period two or more doses of an immediate release reference listed drug (IR RLD) having one half or less of the amount of pseudoephedrine present in the oral composition.

In one embodiment, the above-described method further comprises: D) achieving an AUC_{infinity} for chlorpheniramine in a mammalian subject that is equivalent to an AUC_{infinity} obtained upon administrating over the same time period two or more doses of an immediate release reference listed drug (IR RLD) having one half or less of the amount of pseudoephedrine present in the oral composition.

In another embodiment the present invention relates to a method for achieving AUC_{infinity} values for hydrocodone and pseudoephedrine in a mammalian subject, wherein the method comprises:

(A) administering an extended release (ER) oral pharmaceutical composition comprising: (1) an immediate release (IR) portion consisting of chlorpheniramine and hydrocodone as active ingredients, and (2) an ER portion consisting of chlorpheniramine, hydrocodone and pseudoephedrine as active ingredients,

wherein the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

(B) achieving an AUC_{infinity} for hydrocodone in a mammalian subject that is equivalent to an AUC_{infinity} obtained upon the administration of two doses of an immediate release reference listed drug (IR RLD) having one half the amount of hydrocodone as compared to the oral composition, wherein the oral composition is dosed once, and the IR RLD is dosed twice at zero and six hours, over a 12 hour period, and

C) achieving an AUC_{infinity} for pseudoephedrine in a mammalian subject equivalent to an AUC_{infinity} obtained upon the administration of two doses of an IR RLD having one half the amount of pseudoephedrine as compared to the oral composition, wherein the oral composition is dosed once, and the IR RLD is dosed twice at zero and six hours, over a 12 hour period.

In one embodiment, the above-described method further comprises:

D) achieving an AUC_{infinity} for chlorpheniramine in a mammalian subject equivalent to an AUC_{infinity} obtained upon the administration of two doses of an IR RLD having one half the amount of chlorpheniramine as compared to the oral composition, wherein the oral composition is dosed once, and the IR RLD is dosed twice at zero and six hours, over a 12 hour period.

In yet other embodiments, the present invention envisages a method for providing sufficient AUC_{infinity} of an antihistamine, an antitussive and a decongestant to achieve a therapeutic effect in a human subject for a time period of at least 8 hours after administering a single dose of a non-liquid single drug composition to the human subject, wherein the method comprises: (A) administering to the human subject a single dose of a single non-liquid oral extended-release drug composition comprising chlorpheniramine, hydrocodone and pseudoephedrine as active ingredients, and (B) achieving sufficient AUC_{infinity} of all three active ingredients to observed a therapeutic effect in the human subject over a period of at least 8 hours after the single dose, according to serum analysis.

In a specific embodiment, the present invention envisages a method for providing sufficient AUC_{infinity} of chlorpheniramine, hydrocodone and pseudoephedrine to achieve a therapeutic effect in a human subject for a time period of at least 8 hours after administering a single dose of a single drug composition to the human subject, wherein the method comprises: (A) administering to the human subject a single dose of a single oral extended-release drug composition consisting of chlorpheniramine, hydrocodone and pseudoephedrine as active ingredients, and (B) achieving sufficient AUC_{infinity} of all three active ingredients to observed a therapeutic effect in the human subject over a period of at least 8 hours after the single dose, according to serum analysis.

In one embodiment of the above-described method, the time period of at least 8 hours is 12 hours. In another embodiment, the time period of at least 8 hours is 24 hours.

In some embodiments, the present invention relates to a solid oral extended release drug composition comprising a first portion and a second portion,

wherein the first portion comprises an antihistamine, an antitussive, and optionally a decongestant, as active ingredients in an immediate release form,

wherein the second portion comprises a particulate, pellet, or bead that comprises the antihistamine, the antitussive and the decongestant as three active ingredients in an extended release form,

wherein administration of a single dose of the oral drug composition to a patient provides serum levels of the three active ingredients over a time period of at least 8 hours that are bioequivalent to serum levels achieved upon administration of an appropriate number of doses over the same time period of FDA-approved immediate release reference listed drug (IR RLD) compositions comprising the active ingredients, and

wherein the appropriate number of doses corresponds to a number of doses recommended in one or more FDA-approved labels for the administration of the IR RLD compositions over the same time period.

In other embodiments, the present invention encompasses a method for manufacturing a solid oral extended release combination drug formulation for use in the treatment of symptoms of a cold, flu or allergy, wherein the formulation has reduced abuse potential with regard to any single active ingredient included in the formulation, as compared to an immediate release (IR) formulation comprising the active ingredient, wherein the method comprises:

preparing the solid oral extended release drug composition so that it comprises particulates, pellets, or beads, wherein each particulate, pellet, or bead comprises two or more active ingredients in an extended release form.

In yet another embodiment, the present invention relates to a method for preventing or reducing the ability to extract, isolate or separate out a single active ingredient present in a solid oral extended-release drug composition, comprising:

preparing the solid oral extended release drug composition comprising particulates, pellets, or beads, wherein each particulate, pellet, or bead comprises two or more pharmaceutically acceptable active ingredients in an extended release form.

In some embodiments, the present invention envisages a method for preventing or reducing the ability to extract, isolate or separate out pseudoephedrine or ephedrine present in an non-liquid oral extended-release drug composition, wherein the method comprises preparing the non-liquid oral extended release drug composition so that it comprises particulates, pellets, or beads, wherein each particulate, pellet, or bead comprises the pseudoephedrine or ephedrine, and an antihistamine or an antitussive or both, as active ingredients in an extended release form.

In some other embodiments the present invention relates to a method of reducing the abuse potential of pseudoephedrine or ephedrine present in an non-liquid oral extended-release drug composition, wherein the method comprises preparing the non-liquid oral extended release drug composition so that it comprises particulates, pellets, or beads, wherein each particulate, pellet, or bead comprises an antihistamine, an antitussive, and the pseudoephedrine or ephedrine, as active ingredients in an extended release form.

In another specific embodiment, an non-liquid oral extended release drug composition comprises active ingredients consisting of an antihistamine, an antitussive and a decongestant. In one such embodiment, the immediate release portion of the non-liquid drug composition comprises the active ingredients consisting of an antihistamine, an antitussive and optionally a decongestant. In another such embodiment, the extended release portion of the non-liquid drug composition comprises the active ingredients consisting of an antihistamine, an antitussive and a decongestant.

In yet another specific embodiment, an oral extended release drug composition comprises active ingredients consisting of chlorpheniramine, hydrocodone and pseudoephedrine. In one such embodiment, the immediate release portion of the drug composition comprises the active ingredients consisting of chlorpheniramine, hydrocodone and optionally pseudoephedrine. In another such embodiment, the extended release portion of the drug composition comprises the active ingredients consisting of chlorpheniramine, hydrocodone and pseudoephedrine.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulations or minor changes in experimental design, fall within the scope of the present invention.

### Example 1

Example 1 describes a method of manufacture of “Formulation X.” “Formulation X” is a liquid dispersion of ER coated pellets in syrup intended for the treatment of cough, cold, and allergy symptoms. Formulation X contains 15 mg hydrocodone bitartrate (HC, a centrally-acting antitussive), 120 mg pseudoephedrine hydrochloride (PSE, a sympathomimetic nasal decongestant), and 8 mg chlorpheniramine maleate (CPM, an anti-histamine) in combination per adult dosage (5 ml). In this formulation, the salt forms of the drugs have been used. This formulation was sorted into (4 oz) unit-of-use containers upon manufacture.

Table 1 presents a table outlining an example quantitative composition of Formulation X IR/ER liquid dispersion of extended release pellets in syrup, expressed on a weight basis, in terms of a single 5 ml (6.55 g) dose.

TABLE 1
Quantitative Composition of Formulation X IR/ER Liquid
Dispersion of Extended Release Pellets in Syrup

| Component | Percent Weight |
|---|---|
| Chlorpheniramine Maleate | 0.1382% |
| Pseudoephedrine HCl | 2.073% |
| Hydrocodone Bitartrate | 0.2591% |
| Lactose monohydrate | 1.252% |
| Sodium Alginate | 0.2504% |
| Microcrystalline Cellulose | 8.649% |
| Eudragit RS30D | 3.222% |
| Eudragit RL30D | 0.1342% |
| Triethylcitrate | 0.3504% |
| Talc | 1.043% |
| Titanium Dioxide | 0.2068% |
| FD&C Red #40 Lake | 0.0827% |
| Artificial Strawberry Flavor | 0.3309% |
| Bitter Masking Flavor | 0.2482% |
| Sucralose | 0.08273% |
| Sucrose, NF | 52.86% |
| Propylparaben | 0.01254% |
| Purified Water | 28.81% |
| Total | 100.00% |

The ratio of API concentration in the IR syrup compared to the ER pellet has been designed to provide serum drug bioavailability for 12 hours in a manner that is BE to the immediate release drugs that are currently in the market.

The ratio of API concentration in the IR syrup compared to the ER pellet has been designed to provide serum drug bioavailability for 12 hours in a manner that is BE to the immediate release drugs that are currently in the market.

### Manufacture of Core Pellets for Formulation X

The ER component of Formulation X comprises core pellets. A method for preparing core pellets is described below.

0.207 kg of chlorpheniramine maleate, 4.138 kg of pseudoephedrine hydrochloride, 0.3879 kg of hydrocodone bitartrate, 2.500 kg of lactose monohydrate, 0.5000 kg of sodium alginate and 17.27 kg of microcrystalline cellulose were placed in combination in a high shear mixer. The mixer was operated for 5 minutes with the impeller at 200 RPM and the chopper at 1500 RPM. Subsequent to this powder blending, approximately 10.00 kg of purified water was pumped into the mixer at a rate of approximately 1 L/minute while operating the impeller at 200 RPM and without using the chopper. After all the water was added, wet granulation was continued for 1 minute with the impeller at 200 RPM and the chopper at 1500 RPM. After discharge, the wet mass was left to sit in the open bags for a minimum of 30 minutes.

The resulting wet mass was then extruded using a single screw dome-type extruder with 0.7 mm screen, operating at 45 RPM. Extrusion was continued until the entire quantity of wet mass was processed.

The formation of pellets was accomplished as repetitive batch processes using a spheronizer with a disk having a 3×3 mm truncated pattern operating at approximately 1000 rpm. Nine batches of approximately 3.8 kg each were used with each spheronization run lasting approximately 90 seconds.

The spheronized material was placed in a fluid bed dryer. The beads were dried with an initial inlet air temperature of 60° C., and total air volume of 1500 cubic feet per minute. Subsequent in-process adjustment of inlet air temperature and volume was made to maintain proper fluidization and a product temperature in the range of 50-60° C. Drying was continued until the beads achieved a moisture content of 2% or less. The fluid bed dryer was operated in the cooling mode for sufficient time to bring the product to room temperature, and then the dry beads were discharged.

Sizing of the dry beads was accomplished using an automatic sieve shaker with #22 and #34 screens operating at 1200 RPM. Core pellets passing through the #22 screen and retained on the #34 screen were considered acceptable.

### Coating of the Pellets for Formulation X

A method for coating the core pellets of Formulation X is described below.

18.65 kg of ammonio methacrylate copolymer type B liquid dispersion (Eudragit RS30D) was added through a #20 screen into a stainless steel vessel. 0.7774 kg of ammonio methacrylate copolymer type A liquid dispersion (Eudragit RL30D) was then added through a #20 screen to the same container. The combination of liquid dispersion was mixed using a propeller mixer at approximately 1000 rpm. In a separate container, 0.6087 kg of triethyl citrate and 10.92 kg of purified water were combined. This combination of liquids was also mixed using a propeller mixer at an rpm sufficient to produce a vortex without introducing air into the liquid. While continuing to mix, 1.812 kg of talc was added to the triethyl citrate and water mixture. After mixing for 5 minutes, the stirrer was stopped and removed. The mixture of triethyl citrate, talc and water was transferred into the container with the methacrylate copolymer dispersions with agitation continuing. Sufficient agitation of this coating system was continued throughout the coating operation to maintain a uniform dispersion.

Coating of the core pellets was performed in a Glatt GPCG 30 fluid bed processor with a 12 inch Wurster product container, using a D base plate with a 250 um screen. The column height was set at approximately 2.5 inches from the bottom and a nozzle with a 1 mm opening was employed. 21.75 kg of core beads was placed in the fluid bed processor with initial parameter settings: inlet air temperature target range, 40-50° C.; product temperature target range, 27-32° C., atomization air pressure target range, 1.5-2.5 bar; air volume target range, 250-450 cfm; total air volume, 1500 cfm; filter bag shake interval, 5 seconds every 240 seconds. In process adjustments were made to maintain processing conditions within range. Initial spraying of the coating dispersion was accomplished at a rate of 20 g/minute; as processing continues, the spray rate can eventually be elevated to 100 g/minute.

After all the coating dispersion was applied, the fluid bed processor was operated in cool mode to bring the coated beads to room temperature, and then they were discharged. Subsequently, the coated beads and 75.00 g of talc were blended for 5 minutes and discharged.

Curing of the coated pellets was accomplished in a convection oven. The coated pellets were distributed into trays and placed into the oven. The oven temperature was set for 55° C., and the beads were maintained in the oven for approximately 16 hours. After this time period, the oven was turned off and the pellets were allowed to cool to room temperature.

Sizing of the coated beads was accomplished using an automatic sieve shaker with #16 and #34 screens operating at 1200 RPM. Coated pellets passing through the #16 screen and retained on the #34 screen were considered acceptable.

### Preparing a Vehicle for the Core Pellets of Formulation X

The IR component of Formulation X comprises a liquid, also called a “vehicle” or “vehicle syrup” for Formulation X. A method for making a vehicle syrup is described below.

25.09 kg of purified water was placed in a jacketed stainless steel vessel of sufficient capacity to hold 60 L. A circulating water temperature control system was used to control the temperature of the liquid throughout the operation and a propeller mixer was used to produce agitation. A cover was employed to limit evaporation of water. The water was heated to approximately 70° C. while mixing at a speed such that a vortex was produced without introduction of air into the liquid. 0.01080 kg of propylparaben passed through a #30 screen was added to the water and mixing was continued until the propylparaben completely dissolved. After dissolution of the propylparaben, the temperature control unit was set to 50° C. and 48.00 kg of sucrose was slowly added to the vessel. The vessel was covered and mixing continued until all the sucrose was dissolved.

When the sucrose was completely dissolved, the temperature controller was set to its lowest temperature and the solution was allowed to cool to 25° C. 0.02642 kg of chlorpheniramine maleate, 0.04954 kg of hydrocodone bitartrate, 0.2680 kg of artificial strawberry flavor powder, 0.2160 g of artificial bitter masking powder, and 0.0720 kg of sucralose were added to the syrup. Mixing was continued until all the solid was dissolved. 0.0720 kg of FD&C Red No. 40 aluminum lake and 0.1800 kg of titanium dioxide were then added through a #30 screen into the vessel with stirring continuing until a uniform dispersion was obtained. (Note: This example of the vehicle contains insoluble material which will settle when mixing is stopped.)

Final net weight of the mixture was determined, and, if necessary, purified water was added to compensate for evaporative loss.

### Combining Coated Pellets and Vehicle Syrup for Formulation X

One method for combining the coated pellets and vehicle syrup to form a final Formulation X product is described below.

The vehicle syrup was stirred constantly such that a vortex is formed. After at least 10 minutes of such stirring, 24.00 g of coated beads was added to 130.8 g of vehicle to produce 24 doses or 120 mL of product.

### Example 2

### Effectiveness in Humans

Example 2 describes a study conducted to evaluate effectiveness of Formulation X in humans. Specifically, Example 2 describes a study was conducted to compare a single dose of extended release Formulation X to two doses of immediate release RLDs containing HC, PSE, or CPM used in combination in 16 healthy subjects. One objective of this study was to determine the bioequivalence of two formulations of Formulation X to the corresponding RLDs.

### Absorption

### Hydrocodone

Hydrocodone is well absorbed orally, but undergoes a significant first pass effect involving intestinal and hepatic metabolism. In previously published studies, following a single IR oral dose of 10 mg HC administered to 5 male human subjects, the mean peak serum concentration was 23.6±5.2 ng/mL, with a Tₘₐₓ of approximately 1.3±0.3 hours. “Hycodan®,” available at www.rxmed.com, accessed Jun. 23, 2008; Stout, P.; Farrell, L. Opioids—Effects on Human Performance and Behavior.” Forensic Science Review. 15(1): 29-59 (2003). All hydrocodone metabolites are active, and include hydromorphone, norcodeine, and 6-alpha and 6-beta hydroxy metabolites. Micromedex Health Care Series, DrugDex Evaluations “Hydrocodone Bitartrate/Ibuprofen,” available at http://www.thomsonhc.com/hcs, accessed Jul. 1, 2008, citing Cone, et al “Comparative metabolism of hydrocodone in man, rat, guinea pig, rabbit, and dog,” Drug Metab Dispos 6:488-493 (1978).

Table 2 provides a table comparing parameters, such as AUC_{infinity}, Cₘₐₓ and T_{1/2}, relating to serum levels of hydrocodone (HC) obtained in patients upon administering one dose of “Formulation X” vs two doses of HC reference listed drug (RLD). Treatment A corresponds to one dose of “Formulation X comprising 15 mg HC, 120 mg PSE and 8 mg CPM. Treatment C corresponds to two doses of a cocktail of three single RLDs comprising 7.5 mg HC, 60 mg PSE and 4 mg CPM.

In this study (as shown in Table 2, Treatments A and C), following the administration of Formulation X containing 15 mg HC, 120 mg PSE, and 8 mg CPM to 16 human subjects, the mean peak serum concentration (Cₘₐₓ) of HC was 17.54±4.75 ng/mL, compared to a mean peak serum concentration of 25.64±6.56 ng/mL for the 2 doses of RLD containing 7.5 mg HC each administered at 0 and 6 hours. With Formulation X, a median Tₘₐₓ was 4 hours, compared to a median Tₘₐₓ of 7 hours for the 2 doses of RLD. The difference in Tₘₐₓ for Formulation X, as compared to what is seen in the scientific literature, is due to the fact that the extended release pellets in Formulation X release HC over a period of time to achieve a 12 hour dose. Because two doses of the RLD are administered in this study (as compared to one dose in the previously published studies), however, the peak serum concentration (Cₘₐₓ) achieved with the two doses of RLD is reached at a later time, as compared to that with Formulation X.

TABLE 2
Formulation X vs RLDs - Hydrocodone

| | | | | | Ratio of Geometric Means |
|---|---|---|---|---|---|
| | | | Median | Geometric | [Test/Reference Treatments] |
| Parameter | Treatment | Mean (SD) | Min., Max. | Mean^{a} | Point Estimate (90% CI)^{a} |
| AUC | A | 238.78 (80.93) | 203.18 | 227.09 | [A/C] 0.9328 (0.8482, 1.0258) |
| (ng * hr/mL) | | | 126.1, 393.9 | | |
| | B | 163.43 (57.69) | 146.46 | 155.67 | [B/D] 0.9510 (0.8647, 1.0459) |
| | | | 93.5, 323.7 | | |
| | C | 254.49 (79.97) | 216.28 | 243.46 | — |
| | | | 150.7, 399.9 | | |
| | D | 172.30 (56.65) | 157.23 | 163.69 | — |
| | | | 83.9, 285.1 | | |
| AUC_{LAST} | A | 207.40 (62.44) | 183.10 | 199.11 | [A/C] 0.8559 (0.7780, 0.9416) |
| (ng * hr/mL) | | | 112.7, 327.8 | | |
| | B | 136.22 (34.05) | 128.47 | 132.53 | [B/D] 0.8495 (0.7722, 0.9345) |
| | | | 82.2, 216.5 | | |
| | C | 241.83 (70.46) | 209.61 | 232.64 | — |
| | | | 144.2, 371.3 | | |
| | D | 163.23 (50.19) | 149.28 | 156.01 | — |
| | | | 81.5, 259.0 | | |
| C_{MAX} | A | 17.54 (4.75) | 16.53 | 16.88 | [A/C] 0.6796 (0.6052, 0.7632) |
| (ng/mL) | | | 8.1, 25.1 | | |
| | B | 10.68 (1.97) | 10.45 | 10.51 | [B/D] 0.6472 (0.5763, 0.7268) |
| | | | 7.6, 14.1 | | |
| | C | 25.64 (6.56) | 25.02 | 24.84 | — |
| | | | 13.7, 38.3 | | |
| | D | 16.70 (3.74) | 16.52 | 16.24 | — |
| | | | 7.8, 23.9 | | |
| T_{MAX} (hours) | A | — | 4.00 | — | — |
| | | | 1.0, 9.0 | | |
| | B | — | 6.00 | — | — |
| | | | 1.0, 7.0 | | |
| | C | — | 7.00 | — | — |
| | | | 1.0, 8.0 | | |
| | D | — | 7.00 | — | — |
| | | | 1.0, 9.0 | | |
| T_{1/2} | A | 7.22 (1.45) | 6.79 | — | — |
| (hours) | | | 4.7, 9.2 | | |
| | B | 7.62 (2.34) | 6.93 | — | — |
| | | | 5.0, 13.7 | | |
| | C | 4.38 (0.83) | 4.19 | — | — |
| | | | 3.2, 6.7 | | |
| | D | 4.53 (0.86) | 4.44 | — | — |
| | | | 3.1, 6.1 | | |

Treatment A: Test formulation #1 - Formulation X 1q: 15 mg HC, 120 mg PSE, 8 mg CPM
Treatment B: Test formulation #2 - Formulation X 1q: 10 mg HC, 120 mg PSE, 8 mg CPM
Treatment C: Reference formulation #1 (for Treatment A) - RLD 2q: 7.5 mg HC, 60 mg PSE, 4 mg CPM
Treatment D: Reference formulation #2 (for Treatment B) - RLD 2q: 5 mg HC, 60 mg PSE, 4 mg CPM
AUC = total area under the plasma concentration-time curve from 0 extrapolated to infinity;
AUC_{LAST} = area under the plasma concentration-time curve from 0 to the last quantifiable plasma concentration;
C_{MAX} = maximum observed plasma concentration;
CPM = chlorpheniramine maleate;
HC = hydrocodone bitartrate;
PSE = pseudoephedrine hydrochloride;
T_{MAX} = time of maximum plasma concentration,
T_{1/2} = elimination half-life
^{a}Exponentiated results of analysis of log-transformed values

### Pseudoephedrine

Pseudoephedrine is readily and almost completely absorbed from the GI tract and there is no evidence of first-pass metabolism. In previously published studies, −Tₘₐₓ was observed at 1.5-2.4 hours following administration and bioavailability is the same across formulations and is unaffected by food. Micromedex Health Care Series. DrugDex Evaluations “Pseudoephedrine,” available at http://www.thomsonhc.com/hcs, accessed Jul. 1, 2008; “Common cold and influenza management” MedScape available at www.medscape.com/viewarticle/466063_{—}3, accessed Jun. 26, 2008; Graves D A, et al. “Influence of a standard meal on the absorption of a controlled release pseudoephedrine suspension.” Biopharm Drug Dispos. May-June; 9(3):267-72 (1988).

Table 3 provides a table comparing parameters, such as AUC_{infinity}, Cₘₐₓ and T_{1/2}, relating to serum levels of pseudoephedrine (PSE) obtained in patients upon administering one dose of “Formulation X” vs two doses of PSE RLD. Treatment A corresponds to one dose of “Formulation X comprising 15 mg HC, 120 mg PSE and 8 mg CPM. Treatment C corresponds to two doses of a cocktail of three single RLDs comprising 60 mg PSE, 7.5 mg HC and 4 mg CPM.

In this study (as shown in Table 3, Treatments A and C), following the administration of Formulation X containing 15 mg HC, 120 mg PSE, and 8 mg CPM to 16 human subjects, the mean peak serum concentration (Cₘₐₓ) of PSE was 292.05±49.94 ng/mL, compared to 345.47±78.46 ng/mL after 2 doses of the IR RLD containing 60 mg PSE each. Median Tₘₐₓ was observed 5 hours following dosing of Formulation X, compared to 7 hours following dosing of the RLD. As explained above, the ER pellets in Formulation X release PSE over a period of time to achieve a 12 hour dose. As a result, the peak serum concentration of the RLD in this study is reached at a later time.

TABLE 3
Formulation X vs RLDs - Pseudoephedrine

| | | | | | Ratio of Geometric Means |
|---|---|---|---|---|---|
| | | | Median | Geometric | [Test/Reference Treatments] |
| Parameter | Treatment | Mean (SD) | Min., Max. | Mean^{a} | Point Estimate (90% CI)^{a} |
| AUC | A | 3919.80 | 4203.51 | 3844.66 | [A/C] 0.9545 (0.8855, 1.0288) |
| (ng * hr/mL) | | (769.92) | 2645.4, 4965.8 | | |
| | B | 4042.92 | 4110.85 | 3944.76 | [B/D] 0.9880 (0.9166, 1.0649) |
| | | (969.30) | 2496.4, 6879.8 | | |
| | C | 4149.31 | 3970.85 | 4028.02 | — |
| | | (1034.06) | 2608.5, 6211.7 | | |
| | D | 4107.13 | 4151.74 | 3992.82 | — |
| | | (1013.50) | 2682.8, 6316.5 | | |
| AUC_{LAST} | A | 3291.46 | 3558.28 | 3201.77 | [A/C] 0.9551 (0.8676, 1.0514) |
| (ng * hr/mL) | | (764.26) | 1963.7, 4128.8 | | |
| | B | 3237.90 | 3175.08 | 3111.09 | [B/D] 0.9128 (0.8292, 1.0049) |
| | | (950.08) | 1432.8, 5868.5 | | |
| | C | 3519.47 | 3525.79 | 3352.38 | — |
| | | (1087.08) | 1735.2, 5738.7 | | |
| | D | 3535.29 | 3607.51 | 3408.26 | — |
| | | (979.99) | 1983.6, 5543.1 | | |
| C_{MAX} | A | 292.05 | 288.86 | 287.79 | [A/C] 0.8526 (0.7729, 0.9406) |
| (ng/mL) | | (49.94) | 196.3, 366.2 | | |
| | B | 275.35 | 262.75 | 269.39 | [B/D] 0.7900 (0.7161, 0.8715) |
| | | (60.23) | 171.8, 398.4 | | |
| | C | 345.47 | 331.05 | 337.53 | — |
| | | (78.46) | 216.1, 531.8 | | |
| | D | 347.95 | 354.76 | 341.00 | — |
| | | (69.91) | 219.0, 446.2 | | |
| T_{MAX} (hours) | A | — | 5.00 | — | — |
| | | | 3.0, 10.0 | | |
| | B | — | 5.00 | — | — |
| | | | 3.0, 10.0 | | |
| | C | — | 7.00 | — | — |
| | | | 6.5, 8.0 | | |
| | D | — | 8.00 | — | — |
| | | | 6.5, 9.0 | | |
| T_{1/2} | A | 6.48 | 6.52 | — | — |
| (hours) | | (1.40) | 3.5, 8.4 | | |
| | B | 7.28 | 7.27 | — | — |
| | | (1.73) | 4.5, 11.1 | | |
| | C | 5.06 | 4.96 | — | — |
| | | (0.90) | 3.6, 6.7 | | |
| | D | 4.93 | 4.95 | — | — |
| | | (0.96) | 3.5, 6.8 | | |

Treatment A: Test formulation #1 - Formulation X 1q: 15 mg HC, 120 mg PSE, 8 mg CPM
Treatment B: Test formulation #2 - Formulation X 1q: 10 mg HC, 120 mg PSE, 8 mg CPM
Treatment C: Reference formulation #1 (for Treatment A) - RLD 2q: 7.5 mg HC, 60 mg PSE, 4 mg CPM
Treatment D: Reference formulation #2 (for Treatment B) - RLD 2q: 5 mg HC, 60 mg PSE, 4 mg CPM
AUC = total area under the plasma concentration-time curve from 0 extrapolated to infinity;
AUC_{LAST} = area under the plasma concentration-time curve from 0 to the last quantifiable plasma concentration;
C_{MAX} = maximum observed plasma concentration;
CPM = chlorpheniramine maleate;
HC = hydrocodone bitartrate;
PSE = pseudoephedrine hydrochloride;
T_{MAX} = time of maximum plasma concentration,
T_{1/2} = elimination half-life
^{a}Exponentiated results of analysis of log-transformed values

### Chlorpheniramine

Chlorpheniramine is rapidly and completely absorbed following oral administration. In previously published studies, the drug appeared in the systemic circulation within 30 to 60 minutes and reached Cₘₐₓ in 2 hours, with the concentration decreasing over the next 46 hours. Peets, E et al. “Metabolism of chlorpheniramine maleate in man,” J. Pharmacol. Exp. Ther. 180:464(1972); “Micromedex Health Care Series. DrugDex Evaluations “Chlorpheniramine,” available at http://www.thomsonhc.com/hcs, accessed Jul. 1, 2008. CPM has a 41±16% oral bioavailability, and its absorption, but not its bioavailability, is delayed by food intake. Rumore, M. M, “Clinical pharmacokinetics of chlorpheniramine,” Drug Intel. Clin. Pharm. 18:701-707 (1984). However, CPM appears to undergo substantial metabolism in the GI mucosa during absorption and on first pass through the liver. Limited data indicate that about 25 to 45% of a single oral dose of CPM as a conventional tablet, and 35 to 60% as a solution reaches the systemic circulation as unchanged drug. Limited data also indicate that the bioavailability of extended-release preparations of the drug may be reduced compared with that of conventional tablets or oral solution. Micromedex Health Care Series: Chlorpheniramine, ibid; Therapeutic Goods Administration (Australia) “Core sedating antihistamines product information” available at www.tga.gov.au/npmeds/pi-sedatingantihistamine.rtf, accessed Jun. 26, 2008.

Table 4 provides a table comparing parameters, such as AUC_{infinity}, Cₘₐₓ and T_{1/2}, relating to serum levels of chlorpheniramine (CPM) obtained in patients upon administering one dose of “Formulation X” vs two doses of CPM RLD. Treatment A corresponds to one dose of “Formulation X comprising 15 mg HC, 120 mg PSE and 8 mg CPM. Treatment C corresponds to two doses of a cocktail of three single RLDs comprising 4 mg CPM, 7.5 mg HC and 60 mg PSE.

In this study (as shown in Table 4, Treatments A and C), following the administration of Formulation X containing 15 mg HC, 120 mg PSE, and 8 mg CPM to 16 human subjects, the mean peak serum concentration (Cₘₐₓ) of CPM was 21.20±6.30 ng/mL compared to 28.89±7.92 ng/mL after 2 doses of the RLD containing 4 mg CPM each. The median Tₘₐₓ was achieved at 9 hours following dosing of Formulation X and 9.50 hours following the RLD.

TABLE 4
Formulation X vs RLDs - Chlorpheniramine

| | | | | | Ratio of Geometric Means |
|---|---|---|---|---|---|
| | | Mean | Median | Geometric | [Test/Reference Treatments] |
| Parameter | Treatment | (SD) | Min., Max. | Mean^{a} | Point Estimate (90% CI)^{a} |
| AUC | A | 1217.29 | 942.38 | 881.45 | [A/C] 1.2924 (0.9733, 1.7160) |
| (ng * hr/mL) | (N = 15) | (943.95) | 341.3, 3388.7 | | |
| | B | 770.03 | 610.52 | 694.03 | [B/D] 1.0339 (0.7786, 1.3728) |
| | (N = 14) | (429.72) | 347.4, 1681.5 | | |
| | C | 910.18 | 704.63 | 682.05 | — |
| | | (600.61) | 398.6, 2659.5 | | |
| | D | 964.21 | 732.82 | 671.29 | — |
| | | (756.40) | 347.8, 3316.7 | | |
| AUC_{LAST} | A | 355.69 | 359.71 | 340.95 | [A/C] 0.8457 (0.7564, 0.9454) |
| (ng * hr/mL) | | (112.69) | 228.5, 673.7 | | |
| | B | 329.90 | 293.92 | 314.81 | [B/D] 0.7525 (0.6731, 0.8413) |
| | | (110.92) | 174.2, 576.1 | | |
| | C | 421.31 | 417.25 | 403.17 | — |
| | | (134.24) | 223.2, 753.5 | | |
| | D | 444.78 | 398.39 | 418.32 | — |
| | | (169.87) | 241.3, 914.1 | | |
| C_{MAX} | A | 21.20 | 19.65 | 20.37 | [A/C] 0.7309 (0.6440, 0.8295) |
| (ng/mL) | | (6.30) | 13.8, 33.8 | | |
| | B | 19.18 | 18.57 | 18.35 | [B/D] 0.6395 (0.5635, 0.7258) |
| | | (6.15) | 10.1, 34.8 | | |
| | C | 28.89 | 29.21 | 27.88 | — |
| | | (7.92) | 14.9, 45.4 | | |
| | D | 30.84 | 27.66 | 28.69 | — |
| | | (13.51) | 16.8, 71.4 | | |
| T_{MAX} (hours) | A | — | 9.00 | — | — |
| | | | 4.0, 24.0 | | |
| | B | — | 10.00 | — | — |
| | | | 5.0, 24.0 | | |
| | C | — | 9.50 | — | — |
| | | | 8.0, 12.0 | | |
| | D | — | 9.00 | — | — |
| | | | 8.0, 12.0 | | |
| T_{1/2} | A | 41.71 | 27.50 | — | — |
| (hours) | (N = 15) | (43.33) | 11.3, 181.2 | | |
| | B | 27.50 | 19.84 | — | — |
| | (N = 14) | (18.13) | 12.0, 76.1 | | |
| | C | 19.37 | 17.96 | — | — |
| | | (9.66) | 6.8, 39.0 | | |
| | D | 20.30 | 14.22 | — | — |
| | | (21.08) | 6.2, 96.0 | | |

Treatment A: Test formulation #1 - Formulation X 1q: 15 mg HC, 120 mg PSE, 8 mg CPM
Treatment B: Test formulation #2 - Formulation X 1q: 10 mg HC, 120 mg PSE, 8 mg CPM
Treatment C: Reference formulation #1 (for Treatment A) - RLD 2q: 7.5 mg HC, 60 mg PSE, 4 mg CPM
Treatment D: Reference formulation #2 (for Treatment B) - RLD 2q: 5 mg HC, 60 mg PSE, 4 mg CPM
AUC = total area under the plasma concentration-time curve from 0 extrapolated to infinity;
AUC_{LAST} = area under the plasma concentration-time curve from 0 to the last quantifiable plasma concentration;
C_{MAX} = maximum observed plasma concentration;
CPM = chlorpheniramine maleate;
HC = hydrocodone bitartrate;
PSE = pseudoephedrine hydrochloride;
T_{MAX} = time of maximum plasma concentration,
T_{1/2} = elimination half-life
^{a}Exponentiated results of analysis of log-transformed values

### Elimination and Excretion

### Hydrocodone

The elimination half-life of the parent-compound of hydrocodone is between 3.8-4.5 hours. Micromedex Health Care Series: Hydrocodone. About 70% of total excretion occurring in the first 24 hours after a single oral dose. Id Stout, P.; Farrell, L. “Opioids Effects on Human Performance and Behavior.” Forensic Science Review, 15(1): 29-59. (2003).

In this study (as shown in Table 2, Treatments A and C), the T_{1/2} of Formulation X for HC was measured to be 7.22±1.45 hours, compared to 4.38±0.83 hours for the RLD. This difference was expected because Formulation X is an extended release drug.

### Pseudoephedrine

PSE and its metabolite are excreted in the urine, with up to 90% of a dose being excreted unchanged within 24 hours of dosing. PSE has a half-life of approximately 9-16 hours, which can be affected by urinary pH, prolonging it when alkaline (pH 8) and reducing it when acidic (pH 5). Wishart, D., et al. “DrugBank: a comprehensive resource for in silico drug discovery and exploration,” Nucleic Acids Res. 34: D668-72 (2006); Micromedex Health Care Series: Pseudoephedrine, ibid.

In this study (as shown in Table 3, Treatments A and C), the T_{1/2} of Formulation X for PSE was measured to be 6.48±1.40 hours, compared to 5.06±0.90 hours for the RLD. This difference was expected because Formulation X is an extended release drug.

### Chlorpheniramine

Elimination from the body of chlorpheniramine is primarily by metabolism to monodesmethyl and didesmethyl compounds with up to 26% excreted in the urine. Renal elimination accounts for approximately 50% total excretion with 3%-18% as unchanged drug. Renal excretion increases with increased urine flow and lower pH. See Micromedex Health Care Series: Chlorpheniramine, ibid. Less than 1% is excreted in the feces. The half-life for CPM is 20±5 hours with a measured clearance of 1.7±0.1 mL/min/kg. Rumore, ibid.

In this study (as shown in Table 4, Treatments A and C), the T_{1/2} of Formulation X for CPM was measured to be 41.71±43.33 hours, compared to 19.37±9.66 hours for the RLD. However, the sampling protocol only measured CPM for 24 hours following the administration of Formulation X. Measurement over only 24 hours was probably insufficient to accurately measure the elimination half-life of CPM in Formulation X, and may have contributed to the variability seen in T_{1/2}. It is expected that bioequivalence will be observed.

The data shown in Tables 2-4 do not necessarily reflect the previously published values discussed above regarding Tₘₐₓ and T_{1/2}. The reason for this is that Formulation X is an extended release formulation. The published values for T_{1/2} and Tₘₐₓ are for drug that is immediately available for uptake into the blood and subsequent removal. Formulation X does not release all of the drugs immediately, so Tₘₐₓ is delayed and T_{1/2} and Tₘₐₓ may be shifted because drug is constantly being added for several hours after dosing. Likewise, the RLD values reported in Tables 2-4 do not necessarily reflect previously published values because values in the Tables are for two doses given 6 hours apart instead of the one dose that was used to determine values in the previously published studies. The second dose of the RLDs in the current study causes a second spike in drug concentration. As a result, absolute peak concentration of drug is achieved after the second dosing of the RLD. As a result, the Tₘₐₓ for the RLDs in Tables 2-4 appear delayed, but this is due only to the dosing protocol.

### Bioequivalence of Chlorpheniramine, Hydrocodone and Pseudoephedrine

For a single dose study, the FDA considers AUC to be the only relevant PK parameter for showing BE; however, the FDA does request information on other PK parameters. Tables 2-4 contain the PK data for two different formulations of Formulation X and their respective RLDs. Table 2 shows that for HC, comparing Treatment A (Formulation X comprising 15 mg HC) and Treatment C (RLD comprising 7.5 mg HC, administered two times), the point estimate of AUC for Formulation X was 93.28% of the RLD with a 90% confidence interval (CI) of 84.82% to 102.58%. This meets the FDA's BE standard of 80-125% at 90% CI, and therefore HC achieved bioequivalence in this study. Tables 3 shows that for PSE, comparing Treatment A and Treatment C, the point estimate of AUC for Formulation X was 95.45% of the RLD with a 90% CI of 88.55% to 102.88%, achieving bioequivalence. Tables 4 shows that for CPM, comparing Treatment A and Treatment C, the point estimate of AUC for Formulation X was 129.24% of the RLD with a 90% CI of 97.33% to 171.60%. This value does not established BE. It is expected, however, that the failure to establish BE for CPM in this study was not due to a failure of Formulation X to achieve BE for CPM (in addition to HC and PSE), but instead due to a failure of the current study to account for the long half life of CPM during serum collection. Specifically, when final serum samples were collected, the CPM concentration was not yet decaying to the point that an accurate T_{1/2} could be determined. As a result, the extrapolation required to determine AUC was unreliable. This was true for the CMP RLDs and Formulation X. Future trials will establish that when an appropriate sampling time is used, the CPM data will show that Formulation X is BE to the CPM RLD.

In sum, the 90% confidence limits of AUC for Formulation X fell within 80%425% of the RLDs for HC and PSE, indicating bioequivalence of at least these two drugs. In this particular study, the AUC for CPM did not definitively establish bioequivalence, as the 90% confidence limits for Formulation X, as compared to the CPM RLD, fell between 97.33 to 171.60%. Analysis of CPM AUC was complicated by high intrasubject variability and large differences between AUC_{0-INF} and AUC_{0-LAST} observed for all formulations. A larger study sample to address variability and longer sampling times to account for the long elimination half-life of CPM (20-24 hours by one source) will establish CPM bioequivalence of Formulation X to the CPM RLD. “Drug information: chlorpheniramine,” available at: www.accessmedicine.com/drugs.aspx?index=C, accessed May 15, 2007.

### Analytical HPLC Method for Determining APIs in IR Syrups, ER Beads and IR/ER Suspension of Beads

An analytical method is employed to determine the amount or ratios of APIs that should be used when preparing a formulation comprising an antihistamine, an antitussive and a decongestant as APIs, so that the formulation exhibits extended release (ER) release of all three APIs when administered to a patient.

While the invention has been described and exemplified in sufficient detail for those skilled in this art to make and use it, various alternatives, modifications, and improvements should be apparent without departing from the spirit and scope of the invention. The examples provided herein are representative of certain embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention. As such, the present invention is not to be limited in scope by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

### Example 3

Example 3 describes the results of the in vitro studies conducted to evaluate the release profile of pseudoephedrine, hydrocodone and chlorpheniramine in Formulation X in comparison to release profile of each of these drugs from coated beads comprising chlorpheniramine, hydrocodone and pseudoephedrine in a single bead.

Formulation X was prepared as described in Example 1. Coated beads comprising chlorpheniramine, hydrocodone and pseudoephedrine in a single bead were prepared in accordance with Example 1. The time zero (t=0) sample was analyzed within 1 week from when production was completed; other samples were stored at 25° C. at a chamber with 60% humidity (25/60) and analyzed in 1 month (1 mo), 3 months (3 mo), 6 months (6 mo), 9 months (9 mo) and 12 months (12 mo) from when production was completed, respectively. Assay determinations were made by HPLC. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 5 mL samples at the following time points: 0.5, 4, 8, and 12 hours.

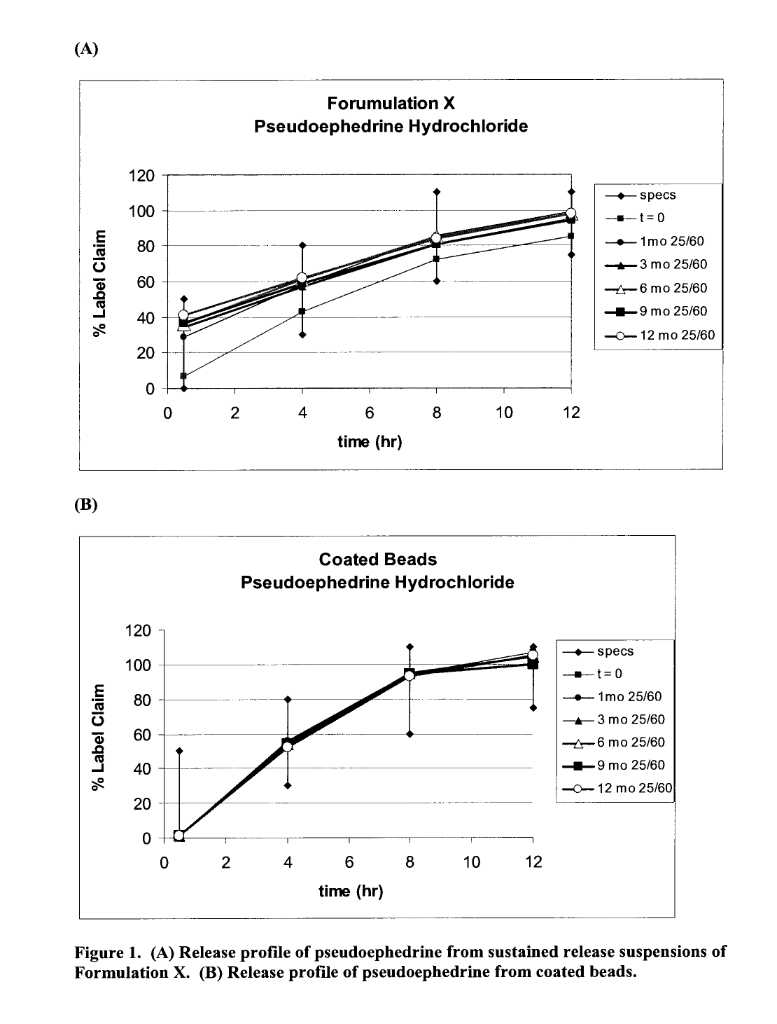

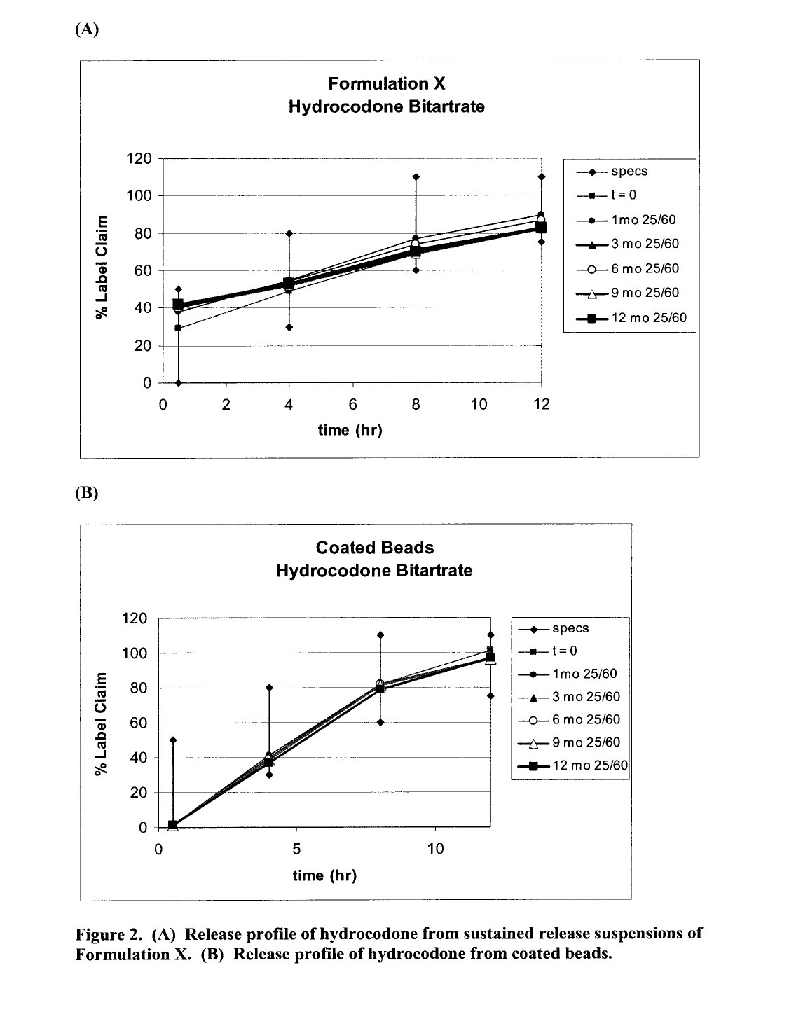

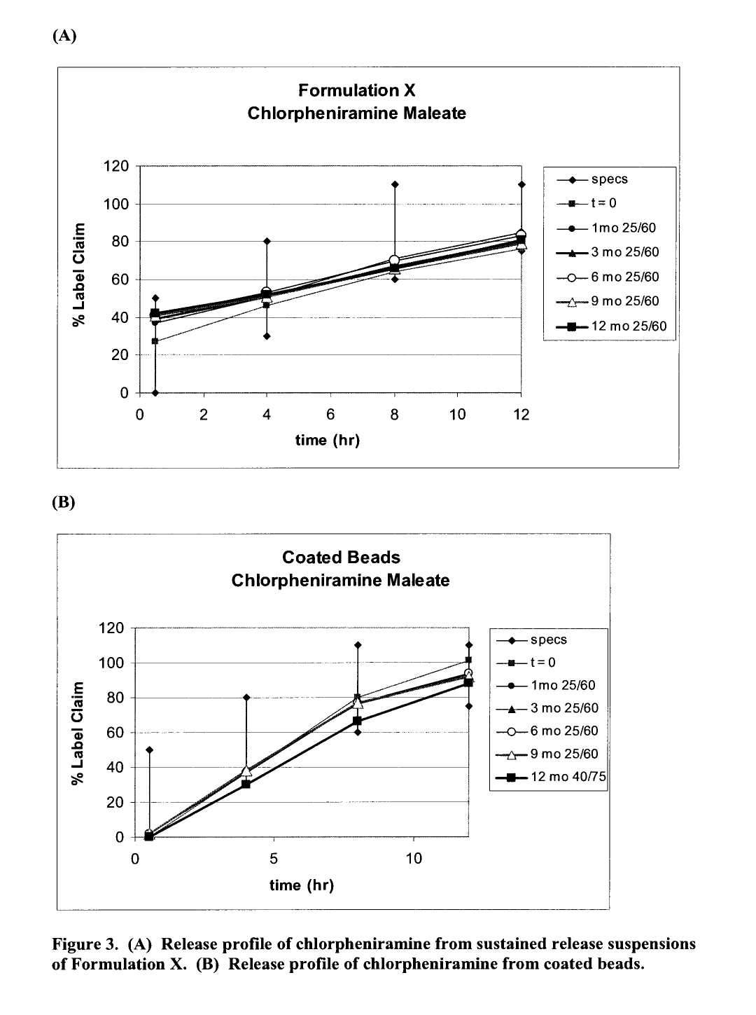

FIG. 1(A) shows the release profile of pseudoephedrine from the liquid form sustained release suspension of Formulation X. FIG. 1(B) shows the release profile of pseudoephedrine from the coated beads. FIG. 2(A) shows the release profile of hydrocodone from the liquid form sustained release suspension of Formulation X. FIG. 2(B) shows the release profile of hydrocodone from the coated beads. FIG. 3(A) shows release profile of chlorpheniramine from the liquid form sustained release suspension of Formulation X. FIG. 3(B) shows the release profile of chlorpheniramine from the coated beads.

FIGS. 1-3 demonstrate that the rate of release of the extended release portion of pseudoephedrine, hydrocodone and chlorpheniramine in Formulation X is comparable to the rate of release of pseudoephedrine, hydrocodone and chlorpheniramine from coated beads when the results are normalized for the presence of an immediate release portion of each of these drugs in Formulation X. The results of the experiment presented in FIGS. 1-3 show that the presence of ionic components, i.e., free drugs in their respective salt forms, in an immediate release phase does not interfere with an adequate rate of sustained release of each of these drugs from the extended release portion of Formulation X. FIGS. 1-3 also demonstrate that the rate of release of the extended release portion of pseudoephedrine, hydrocodone and chlorpheniramine in Formulation X tested at time zero is comparable with the rate of release of the extended release portion of pseudoephedrine, hydrocodone and chlorpheniramine in Formulation X stored for 1 month, 3 months, 6 months, 9 months and 12 months post production. Thus, the results of the experiment presented in FIGS. 1-3 also show that Formulation X maintains stability and an adequate rate of sustained release of each of these drugs after storage of Formulation X at room temperature conditions for 1 month, 3 months, 6 months, 9 months and 12 months.

### Example 4

Example 4 describes the results of in vitro studies conducted to compare the release profile of a liquid form controlled release composition comprising base forms of drugs in the dispersed phase with the release profile of a liquid form controlled release composition comprising salt forms of drugs in the dispersed phase.

The experiment was carried out with two types of compositions, each designed to deliver the equivalent of 30 mg of pseudoephedrine hydrochloride (7.5 mg IR and 22.5 mg ER), 7.5 mg of hydrocodone bitartrate (1.87 IR and 5.63 mg ER), and 6 mg of chlorpheniramine maleate (1.5 mg IR and 4.5 mg ER) in 5 ml of the liquid dosage form. The first type contained 18.4 mg of pseudoephedrine, 3.41 mg of hydrocodone and 1.58 mg of chlorpheniramine, wherein the drugs were used in their base forms, bound to the alginic acid resin. The drug-loaded alginate beads were prepared, coated and dispersed into a dispersion medium containing 65% w/w of sucrose. The second type contained 22.5 mg of pseudophedrine hydrochloride, 5.62 mg of hydrocodone bitartrate and 4.5 mg of chlorpheniramine maleate, bound to sodium alginate. In the second type composition, the core beads were manufactured and coated essentially as described in Example 1, and such coated beads were dispersed into a dispersion medium containing 65% w/w of sucrose. In both cases, the dispersion medium contained the portion of each dose of the salt forms of the drugs designed for immediate release.

The samples were stored at room temperature for 3 weeks prior to analysis. Then, assay determinations were made by HPLC. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 1 mL samples at the following time points: 5, 30, 60, 90 minutes; 4, 8, 12, 16, 20, 24 hours.

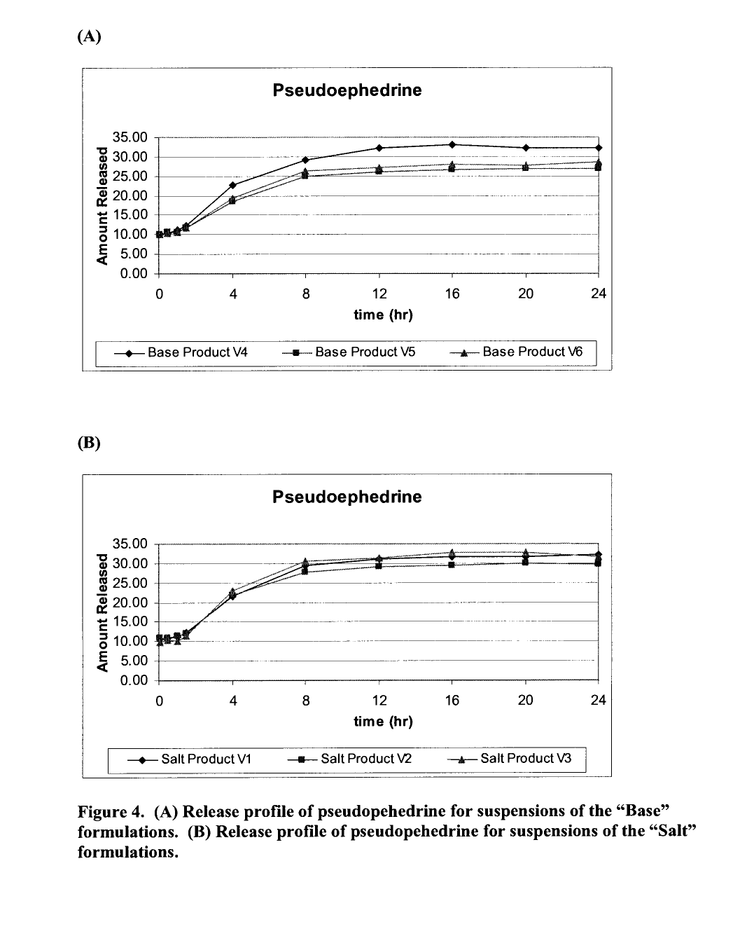

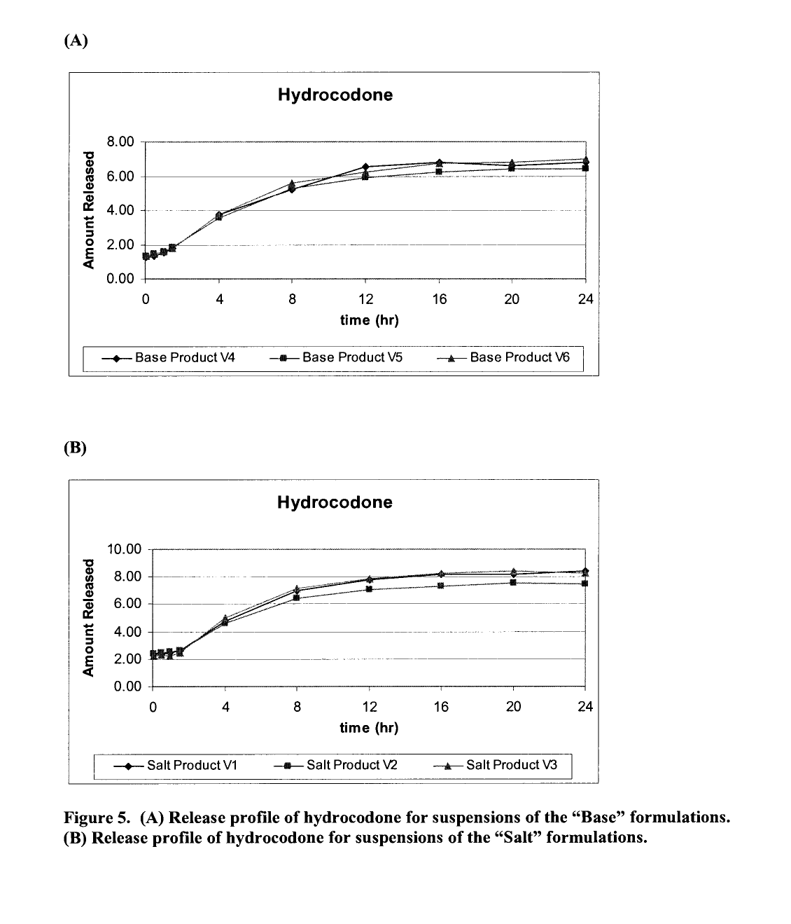

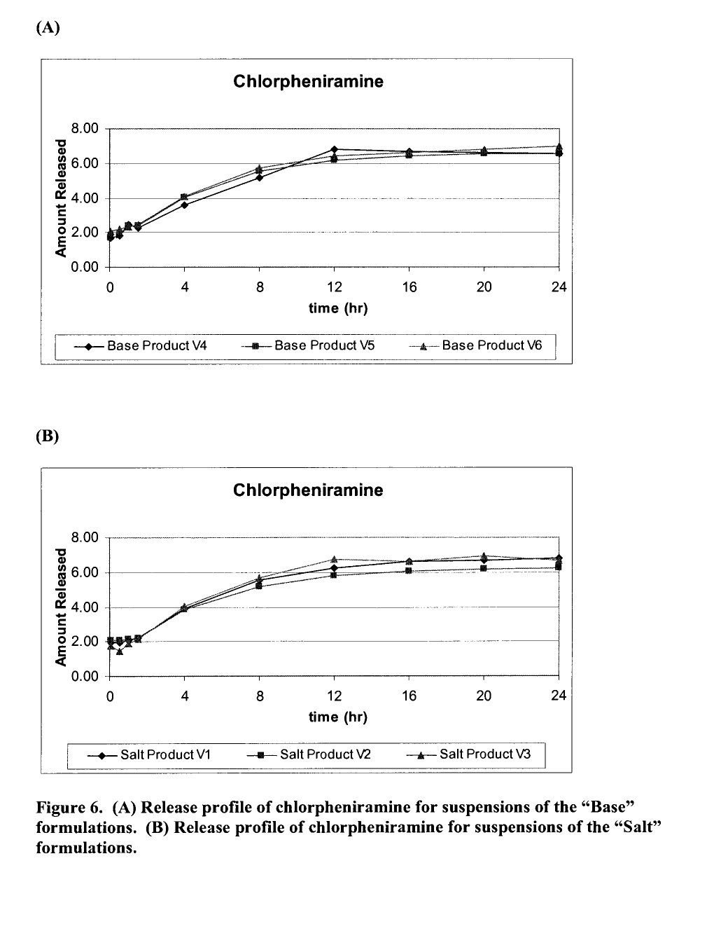

FIG. 4(A) shows the release profile of base formulations of pseudoephedrine. FIG. 4(B) shows the release profile of salt formulations of pseudoephedrine. FIG. 5(A) shows the release profile of base formulations of hydrocodone. FIG. 5(B) shows the release profile of salt formulations of hydrocodone. FIG. 6(A) shows the release profile of base formulations of chlorpheniramine. FIG. 6(B) shows the release profile of salt formulations of chlorpheniramine.

FIGS. 4-6 show that, in a liquid form sustained release compositions, the rate of release of salt forms of pseudoephedrine, hydrocodone and chlorpheniramine is comparable to the rate of release of base forms of pseudoephedrine, hydrocodone and chlorpheniramine, respectively. Specifically, these drugs in salt and base forms show about the same or not significantly different release characteristics at each time point of the experiment. The results of the experiment presented in FIGS. 4-6 show that salt forms of drugs may be used in liquid form controlled release compositions of the invention to achieve adequate sustained release profile of such drugs.

### Example 5

Example 5 shows the release profile of hydrocodone from a liquid form sustained release composition comprising only one active ingredient, hydrocodone, in the dispersed phase.

The experiment was carried out using a liquid form controlled release composition comprising 10 mg of hydrocodone base bound to alginic acid matrix. The hydrocodone alginate beads were prepared to contain 10 mg hydrocodone bound to alginic acid and 20% lactose monohydrate. Table 5 outlines quantitative composition of the Hydrocodone Alginate Bead formulation.

TABLE 5
Hydrocodone Alginate Bead Formulation

| Component | Weight |
|---|---|
| Batch Size | 250 g |
| Hydrocodone base | 5 g |
| Alginic acid | 5 g |
| Lactose (20%) | 50 g |
| Avicel PH101 | 190 g |

Hydrocodone Alginate Beads were then coated with 1.5% Opadry/31.5% Eudragit RS30D (on a weight by weight basis). The coated hydrocodone alginate beads were dispersed in 5 mL of Syrup per 10 mg of hydrocodone. Table 6 outlines quantitative composition of the liquid dispersion of extended release pellets containing hydrocodone.

TABLE 6
Quantitative Composition of the Hydrocodone formulation:
Liquid Dispersion of Extended Release Pellets in Syrup

| Component | Percent Weight |
|---|---|
| Hydrocodone | 0.1376% |
| Alginic Acid | 0.1376% |
| Lactose Monohydrate | 1.376% |
| Avicel PH101 | 5.230% |
| Opadry Clear | 0.1541% |
| Eudragit RS30D | 2.915% |
| Triethylcitrate | 0.3241% |
| Sucrose | 58.14% |
| Purified Water | 31.58% |

The samples were assayed immediately after production was completed. Assay determinations were made by HPLC. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 1 mL samples at the following time points: 30 minutes, 1 hour, 1.5 hours, 2 hours, 3 hours, 4 hours, 6 hours, 8 hours and 10 hours.

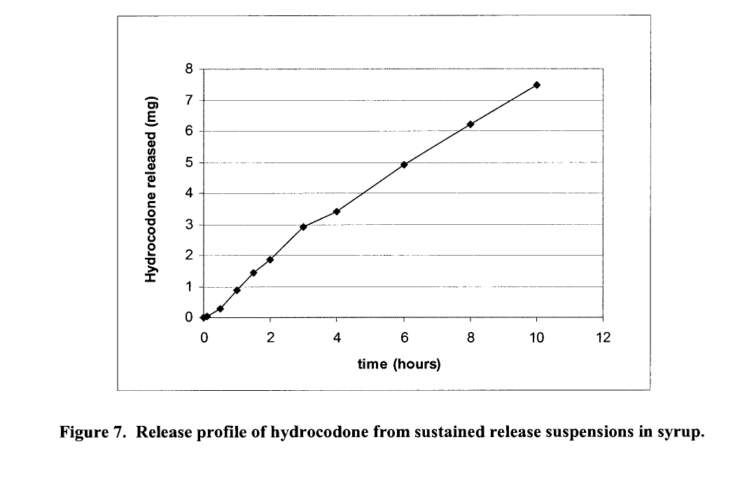

FIG. 7 shows release profile of hydrocodone from a liquid form controlled release composition comprising hydrocodone bound to alginic acid matrix.

FIG. 7 shows the rate of release of hydrocodone from a liquid form sustained release composition comprising beads containing only one active ingredient, hydrocodone, bound to an ion-exchange matrix and dispersed in Syrup.

1. An oral extended release drug composition in a non-liquid form comprising a first portion and a second portion, wherein
the first portion comprises active ingredients consisting of chlorpheniramine, hydrocodone, and optionally pseudoephedrine in an immediate release form,
the second portion comprises a particulate, pellet, or bead that comprises active ingredients consisting of chlorpheniramine, hydrocodone and pseudoephedrine in an extended release form,
administration of a single dose of the oral drug composition to a patient provides serum levels of the three active ingredients over a time period of at least 8 hours that are bioequivalent to serum levels achieved upon administration of an appropriate number of doses over the same time period of FDA-approved immediate release reference listed drug (IR RLD) compositions comprising the active ingredients, and
the appropriate number of doses corresponds to a number of doses recommended in one or more FDA-approved labels for the administration of the IR RLD compositions over the same time period.

2. The drug composition of claim 1, wherein the drug composition is in an oral solid form.

3. The drug composition of claim 1, wherein the drug composition is in an oral capsule form.

4. A method for treating coughing, symptoms of coughing, nasal discharge, congestion or sneezing associated with a cold, flu or an allergy for a time period of at least 8 hours, comprising administering to a human subject in need of such a treatment a single dose of the drug composition of claim 1 effective to treat coughing, symptoms of coughing, nasal discharge, congestion or sneezing associated with a cold or an allergy, for the time period of at least 8 hours.

5. An oral pharmaceutical formulation in a non-liquid form comprising active ingredients consisting of chlorpheniramine, hydrocodone and pseudoephedrine, wherein the formulation exhibits immediate release (IR) and extended release (ER) of the active ingredients, wherein
the formulation comprises an immediate release portion and an extended release portion, and
administration of a single dose of the oral formulation to a patient provides serum levels of chlorpheniramine, hydrocodone and pseudoephedrine over a time period of at least 8 hours that are bioequivalent to serum levels achieved upon administration of two or more doses, over the same time period, of one or more IR compositions comprising chlorpheniramine, hydrocodone and/or pseudoephedrine.

6. A method of making the oral pharmaceutical formulation of claim 5, comprising preparing the immediate release portion, wherein the immediate release portion comprises active ingredients consisting of chlorpheniramine and hydrocodone, but not pseudoephedrine.

7. A method of making the oral pharmaceutical formulation of claim 5, comprising preparing the extended release portion, which comprises preparing particulates, pellets or beads, wherein each individual particulate, pellet or bead comprises active ingredients consisting of chlorpheniramine, hydrocodone and pseudoephedrine,
wherein the method further comprises combining the extended release portion with the immediate release portion.

8. The method of claim 7, wherein the method further comprises coating the particulates, pellets or beads with a membrane coating prior to combining the extended release portion with the immediate release portion.

9. An oral extended release drug composition in a non-liquid form comprising a first portion and a second portion, wherein
the first portion comprises active ingredients consisting of chlorpheniramine, hydrocodone, and optionally pseudoephedrine in an immediate release form,
the second portion is a particulate, pellet or bead that comprises active ingredients consisting of chlorpheniramine, hydrocodone and pseudoephedrine in an extended release form,
administration of a sufficient number of doses of the drug composition to a patient to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release drug compositions comprising the active ingredients, and
the appropriate number of doses corresponds to a number of doses recommended in one or more FDA-approved labels for the administration of the one or more FDA-approved immediate release drug compositions over the same time period.

10. An oral extended release drug composition in a non-liquid form comprising a first portion and a second portion, wherein
the first portion comprises active ingredients consisting of chlorpheniramine, hydrocodone, and optionally pseudoephedrine in an immediate release form,
the second portion comprises a particulate, pellet, or bead that comprises active ingredients consisting of chlorpheniramine, hydrocodone and pseudoephedrine in an extended release form,
administration of a single dose of the drug composition to a patient provides serum levels of the three active ingredients over a time period of at least 8 hours that are bioequivalent to serum levels achieved upon administration of an appropriate number of doses over the same time period of an FDA-approved immediate release reference listed drug (IR RLD) composition comprising all three active ingredients, and
the appropriate number of doses corresponds to a number of doses recommended in an FDA-approved label for the administration of the IR RLD composition over the same time period.

11. An oral pharmaceutical composition in a non-liquid form comprising: (1) an immediate release (IR) portion comprising active ingredients consisting of chlorpheniramine and hydrocodone, and (2) an extended release (ER) portion comprising active ingredients consisting of chlorpheniramine, hydrocodone and pseudoephedrine, wherein
the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,
administration of a single dose of the oral composition provides an AUC_{Infinity} for hydrocodone in a human subject that is equivalent to an AUC_{infinity} obtained upon administration of two or more doses of an immediate release reference listed drug (IR RLD) having one half or less of the amount of hydrocodone present in the oral composition, and
administration of a single dose of the oral composition provides an AUC_{Infinity} for pseudoephedrine in a human subject that is equivalent to an AUC_{infinity} obtained upon administration of two or more doses of an immediate release reference listed drug (IR RLD) having one half or less of the amount of pseudoephedrine present in the oral composition.

12. The oral composition of claim 11, wherein administration of a single dose of the oral composition provides an AUC_{infinity} for chlorpheniramine in a human subject that is equivalent to an AUC_{infinity} obtained upon administration of two or more doses of an immediate release reference listed drug (IR RLD) having one half or less of the amount of chlorpheniramine present in the oral composition.

13. An oral pharmaceutical composition in a non-liquid form comprising: (1) an immediate release (IR) portion comprising active ingredients consisting of chlorpheniramine and hydrocodone, and (2) an extended release (ER) portion comprising active ingredients consisting of chlorpheniramine, hydrocodone and pseudoephedrine, wherein
the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,
the oral composition demonstrates an AUC_{infinity} for hydrocodone in a human subject that is equivalent to an AUC_{infinity} obtained upon administration of two doses of an immediate release reference listed drug (IR RLD) having one half the amount of hydrocodone as compared to the oral composition, wherein the oral composition is dosed once, and the IR RLD is dosed twice at zero and six hours, over a 12 hour period, and
the oral composition demonstrates an AUC_{infinity} for pseudoephedrine in a human subject equivalent to an AUC_{infinity} obtained upon administration of two doses of an IR RLD having one half the amount of pseudoephedrine as compared to the oral composition, wherein the oral composition is dosed once, and the IR RLD is dosed twice at zero and six hours, over a 12 hour period.

14. The oral composition of claim 13, wherein the oral composition demonstrates an AUC_{infinity} for chlorpheniramine in a human subject equivalent to an AUC_{infinity} obtained upon administration of two doses of an IR RLD having one half the amount of chlorpheniramine as compared to the oral composition, wherein the oral composition is dosed once, and the IR RLD is dosed twice at zero and six hours, over a 12 hour period.

15. An oral extended-release drug composition in a non-liquid form comprising active ingredients consisting of chlorpheniramine, hydrocodone and pseudoephedrine, wherein the composition provides sufficient AUC_{infinity} of all three active ingredients to achieve a therapeutic effect for a time period of at least 8 hours after a single dose in a human subject, according to serum analysis.

16. A method for preventing or reducing an ability to extract, isolate or separate out pseudoephedrine present in an oral extended-release drug composition, comprising:
preparing the oral extended release drug composition so that it comprises active ingredients consisting of chlorpheniramine, hydrocodone and pseudoephedrine, wherein said drug composition comprises a first portion and a second portion, wherein
the first portion comprises the active ingredients consisting of chlorpheniramine, hydrocodone , and optionally pseudoephedrine in an immediate release form, and
the second portion comprises particulates, pellets, or beads, wherein each particulate, pellet, or bead comprises the active ingredients consisting of chlorpheniramine, hydrocodone and pseudoephedrine in an extended release form, and
preventing or reducing the ability to extract, isolate or separate out pseudoephedrine present in an oral extended-release drug composition.

17. The method of claim 16, wherein the method further comprises preventing or reducing an ability to extract, isolate or separate out hydrocodone present in the oral extended-release drug composition.

18. The method of claim 16, further comprising a step of manufacturing that makes extraction, isolation or separation of the pseudoephedrine from the oral extended-release drug composition more difficult, as compared to an immediate release composition comprising pseudoephedrine.

19. A method of reducing the abuse potential of pseudoephedrine present in an oral extended-release drug composition, comprising:
preparing the oral extended release drug composition so that it comprises active ingredients consisting of chlorpheniramine, hydrocodone and pseudoephedrine, wherein said drug composition comprises a first portion and a second portion, wherein
the first portion comprises the active ingredients consisting of chlorpheniramine, hydrocodone, and optionally pseudoephedrine in an immediate release form, and
the second portion comprises a particulate, pellet, or bead that comprises the active ingredients consisting of chlorpheniramine, hydrocodone, and pseudoephedrine in an extended release form.

20. A method for reducing the abuse potential of hydrocodone or pseudoephedrine present in an oral extended-release drug composition, comprising preparing the oral extended release drug composition so that it comprises active ingredients consisting of chlorpheniramine, hydrocodone and pseudoephedrine, wherein said drug composition comprises (a) a particulate, pellet, or bead comprising the active ingredients consisting of chlorpheniramine, hydrocodone and pseudoephedrine in an extended release form, and (b) the active ingredients consisting of chlorpheniramine, hydrocodone, and optionally pseudoephedrine in an immediate release form.

21. A method for preventing or reducing the ability to extract, isolate or separate out pseudoephedrine present in an oral extended-release drug composition, wherein the method comprises preparing the oral extended release drug composition so that it comprises active ingredients consisting of chlorpheniramine, hydrocodone and pseudoephedrine, wherein said drug composition comprises (a) particulates, pellets, or beads, wherein each particulate, pellet, or bead comprises the active ingredients consisting of chlorpheniramine, hydrocodone and pseudoephedrine in an extended release form, and (b) the active ingredients consisting of chlorpheniramine, hydrocodone, and optionally pseudoephedrine in an immediate release form.

22. A method of reducing the abuse potential of pseudoephedrine present in an oral extended-release drug composition, wherein the method comprises preparing the oral extended release drug composition so that it comprises active ingredients consisting of chlorpheniramine, hydrocodone and pseudoephedrine, wherein said drug composition comprises (a) particulates, pellets, or beads, wherein each particulate, pellet, or bead comprises the active ingredients consisting of chlorpheniramine, hydrocodone, and the pseudoephedrine in an extended release form, and (b) the active ingredients consisting of chlorpheniramine, hydrocodone, and optionally pseudoephedrine in an immediate release form.
